(19) United States
(12) Patent Application Publication
(10) Pub. No.: US 2026/0368892 A1
(43) Pub. Date: Nov. 19, 2026

(54) Diagnostic and Prognostic Modeling of Stroke using Fuzzy Neural Networks

(71) Applicant: REGENTS OF THE UNIVERSITY OF MICHIGAN, Ann Arbor, MI (US)

(72) Inventors: Emily Wittrup, Ann Arbor, MI (US); Kayvan Najarian, Northville, MI (US)

(21) Appl. No.: 19/677,615

(22) Filed: May 14, 2026

Related U.S. Application Data
(60) Provisional application No. 63/805,631, filed on May 14, 2025.

(51) Int. Cl. G06T 7/00; G16H 10/60; G16H 30/40; G16H 50/20

(52) U.S. Cl. CPC G06T 7/0012; G16H 10/60; G16H 30/40; G16H 50/20; G06T 2207/10088; G06T 2207/20081; G06T 2207/20084

(57) ABSTRACT

Methods and systems for assessing medical imaging data for a patient using a trained fuzzy ruleset are disclosed herein. A method includes: generating a cerebrovascular accident ruleset based on fuzzy concepts representative of clinician expertise data, the clinician expertise data associated with cerebrovascular accident procedures; initializing a fuzzy machine learning model based on the cerebrovascular accident ruleset; training the fuzzy machine learning model, on historical electronic health record data and medical image embeddings generated via one or more trained autoencoders, to generate a trained fuzzy ruleset; and applying the trained fuzzy ruleset to the at least one embedding vector and the contextual information to generate one or more of: (i) an intervention recommendation for the patient, or (ii) a stroke outcome classification for the medical imaging data.

### CROSS-REFERENCE TO RELATED APPLICATIONS

This application claims the priority benefit under 35 U.S.C. § 119 (e) of U.S. Provisional Application No. 63/805,631, filed May 14, 2025, which is incorporated herein by reference in its entirety.

### FIELD OF THE DISCLOSURE

The present disclosure generally relates to using explainable machine learning (ML) to predict outcomes following a cerebrovascular accident (stroke) and, more particularly, to techniques for evaluating medical imaging data for a patient using various machine learning models to generate human interpretable outcome classification(s) and/or recommendation(s) from the medical imaging and clinical data.

### BACKGROUND

The background description provided herein is for the purpose of presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Machine learning (ML) and artificial intelligence (AI) models are used by professionals in a wide variety of fields to analyze massive quantities of data and make determinations based on said data. To properly analyze the data and/or make determinations, the ML and AI models often rely on rules programmed into the models in question. However, high performance models that can analyze complicated problems are often difficult for a user to understand. Notably, such “black box” models lack transparency and justification of the output recommendations and/or analysis, especially for the average individual user, who is often not trained in ML and/or AI programming.

Moreover, present ML and AI models require precise annotated data for training and cannot properly analyze or use the same approximate or “fuzzy” rules that human experts do. Many ML and AI models, for example, cannot properly determine that a parameter is “a little high” the same way a human expert would. As such, an interpretable model for machine learning and/or artificial intelligence with a mechanism to leverage approximate knowledge for formation or training of the model and/or rulesets is desired.

Cerebrovascular accidents (strokes) are independently associated with significant morbidity and mortality. Additionally, duration and completion of recovery from a cerebrovascular accident may vary significantly between patients. Typically, medical imaging data (e.g., magnetic resonance imaging data, computed tomography imaging data, etc.), for a patient that has experienced a cerebrovascular accident, is evaluated by a clinician. However, such evaluations are highly variable between providers and centers, and may lead to erroneous diagnosis, classifications, treatment recommendations, projected prognosis, etc. Due to the complexity of stroke management/intervention and the high stakes involved, present ML and AI models are not suitable for providing diagnosis, classifications, recommendations, etc., based on medical imaging data. Additionally, present ML and AI models are not equipped to provide interpretable models that allow clinicians to review and oversee the generation of classifications and recommendations.

### SUMMARY

In one embodiment, a computer-implemented method for assessing medical imaging data for a patient using a trained fuzzy ruleset may be provided. The computer-implemented method may be implemented via one or more local or remote processors, servers, sensors, transceivers, memory units, and/or other electronic or electrical components. The computer-implemented method may include: (1) generating, via one or more processors, a cerebrovascular accident ruleset based on fuzzy concepts, wherein the cerebrovascular accident ruleset represents clinician expertise data associated with cerebrovascular accident procedures for a patient population; (2) initializing, via the one or more processors, a fuzzy machine learning model based on the cerebrovascular accident ruleset; (3) training, via the one or more processors, the fuzzy machine learning model on historical electronic health record (EHR) data for the patient population to generate a trained fuzzy ruleset; (4) generating, via one or more trained autoencoders, at least one embedding vector for the medical imaging data; (5) generating, via the one or more processors, contextual information for the medical imaging data based on the historical EHR data for the patient population; and (6) applying, via the one or more processors, the trained fuzzy ruleset to the at least one embedding vector and the contextual information to generate one or more of: (i) an intervention recommendation for the patient, and (ii) a stroke outcome classification for the medical imaging data.

In another embodiment, a system for assessing medical imaging data for a patient using a trained fuzzy ruleset may be provided. The system may include: one or more processors; one or more memories including computer-executable instructions that, when executed by the one or more processors, cause the computing system to: (1) generate a cerebrovascular accident ruleset based on fuzzy concepts, wherein the cerebrovascular accident ruleset represents clinician expertise data associated with cerebrovascular accident procedures for a patient population; (2) initialize a fuzzy machine learning model based on the cerebrovascular accident ruleset; (3) train the fuzzy machine learning model on historical electronic health record (EHR) data for the patient population to generate a trained fuzzy ruleset; (4) generate, via one or more trained autoencoders, at least one embedding vector for medical imaging data of a patient; (5) generate contextual information for the medical imaging data based on the historical EHR data for the patient population; and (6) apply the trained fuzzy ruleset to the at least one embedding vector and the contextual information to generate one or more of: (i) an intervention recommendation for the patient, and (ii) a stroke outcome classification for the medical imaging data.

In yet another embodiment, a tangible, non-transitory computer readable medium for assessing medical imaging data for a patient using a trained fuzzy ruleset may be provided. The tangible, non-transitory computer readable medium may include computer-executable instructions that, when executed by one or more processors of a computing system, cause the computing system to: (1) generate a cerebrovascular accident ruleset based on fuzzy concepts, wherein the cerebrovascular accident ruleset represents clinician expertise data associated with cerebrovascular accident procedures for a patient population; (2) initialize a fuzzy machine learning model based on the cerebrovascular accident ruleset; (3) train the fuzzy machine learning model on historical electronic health record (EHR) data for the patient population to generate a trained fuzzy ruleset; (4) generate, via one or more trained autoencoders, at least one embedding vector for medical imaging data of a patient; (5) generate contextual information for the medical imaging data based on the historical EHR data for the patient population; and (6) apply the trained fuzzy ruleset to the at least one embedding vector and the contextual information to generate one or more of: (i) an intervention recommendation for the patient, and (ii) a stroke outcome classification for the medical imaging data.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 illustrates a block diagram of an example logic circuit for implementing example methods and/or operations for assessing medical imaging data for a patient using a trained fuzzy ruleset, in accordance with some embodiments described herein.

FIG. 2 illustrates a block diagram depicting an example application architecture for training and/or applying a model using a tropical geometry-based interpretable machine learning method, in accordance with some embodiments described herein.

FIG. 3A illustrates a diagram depicting methods for receiving an input dataset and extracting rules therefrom as well as for receiving a trained model and dataset and subsequently developing a summarized ruleset, implemented by the application of FIG. 2, in accordance with some embodiments described herein.

FIG. 3B illustrates a diagram depicting further systems and methods for receiving an input dataset and extracting rules therefrom, implemented by the application of FIG. 2, in accordance with some embodiments described herein.

FIG. 4 illustrates a block-flow diagram depicting an example method for generating a predicted outcome following a cerebrovascular accident, implemented in the computing environment of FIG. 1, in accordance with some embodiments described herein.

FIG. 5 illustrates a block-flow diagram depicting an example architecture for an ensemble machine learning model, in accordance with some embodiments described herein.

FIG. 6A illustrates an example visualization of rulesets and concepts, as well as contributions of the rulesets to positive classes, as may be presented via an interactive user interface of a computing device, in accordance with some embodiments described herein.

FIG. 6B illustrates a graphical depiction of an example classification visualization, in accordance with some embodiments described herein.

FIG. 6C illustrates a graphical depiction of an example classification visualization, in accordance with some embodiments described herein.

FIG. 6D illustrates a graphical depiction of an example classification visualization, in accordance with some embodiments described herein.

FIG. 7 illustrates a flowchart depicting an example method for assessing medical imaging data for a patient using a trained fuzzy ruleset, implemented in the computing environment of FIG. 1, in accordance with some embodiments described herein.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

When evaluating or describing concepts and/or characteristics, human actors use fuzzy descriptions without a strict definition. Such practices are true even for experts making difficult and/or complicated evaluations. For example, a doctor evaluating a patient heart rate may note that the heart rate is “a little high” without explicitly defining the boundaries of what heart rate range constitutes “a little high.” Another expert or, depending on the situation, a layperson may understand the evaluation despite the lack of hard numbers. Similarly, a stockbroker may describe a particular stock as “low performing” or a network analyst may describe security as “light”, and another evaluator would have a rough idea as to what the expert means despite the lack of precision. In medical environments, for example, clinical guidelines typically have hard thresholds (e.g., if blood pressure of a patient exceeds a threshold, then the patient is likely hypertensive), however, such unforgiving thresholds pose significant issues (e.g., the patient is hypertensive despite the blood pressure of the patient not exceeding the threshold).

However, computers using standard analysis techniques cannot understand the intuitive fuzzy concepts that human experts may understand naturally, leading to disconnects between the computers and the human experts using the computers. Implementing fuzzy concepts into computer-understandable techniques leads to algorithms and functions that are slow or inaccurate. Using piecewise functions to directly represent the fuzzy concepts that are more intuitive to humans provides better accuracy. However, such functions cannot be analyzed and/or improved using standard techniques, such as gradient-based techniques, which are only usable for differentiable functions, but guarantee a convergence. For example, Gaussian functions are a common class of differentiable functions utilized in place of such piecewise functions for gradient-based techniques. The use of such Gaussian functions, however, loses accuracy. Similarly, available techniques for sharper, piecewise functions, such as genetic algorithms, also lack in speed and/or accuracy. Combinatorial or exhaustive search techniques, for example, are often implemented for encoding input data for such functions and require extended periods of time to approach any sustainable level of accuracy. Similarly, techniques besides gradient techniques may not guarantee convergence, or may take prohibitive quantities of time to do so.

By using tropical geometry, a system may approximate the sharp piecewise functions with smooth continuous approximations. In some implementations, the system may approximate the smooth continuous approximations such that the smooth approximations are infinitely close to the sharp edges while still being continuous. Similarly, the system may take data from any number of sources to clarify how to encode various fuzzy concepts and how to determine which concepts and/or variables are relevant to a final determination.

By using tropical geometry to approximate fuzzy concepts, a system can generate and/or pull rules from a model and train the rules to improve the general accuracy and speed of making determinations. Similarly, because the concepts are fuzzy, the model is transparent and easy for humans to understand, unlike standard techniques, in which the determinations are oblique and/or difficult for even experts to understand.

Further, a system using tropical geometry to approximate fuzzy concepts can (i) improve good rules using input data, (ii) create rules from only input data, or (iii) determine that a ruleset is incomplete and/or incorrect. As such, the system can remove irrelevant rules or generating new rules to improve the overall determination made by an expert or individual assessing or evaluating a condition. Similarly, such a system may present combinations of rules and/or variables that would normally be overlooked or missed by traditional systems that rely on receiving rules to train. Moreover, incorporating good rules into a model reduces the amount of training data needed to achieve a given level of accuracy in performance, improving the overall system performance.

Moreover, because the techniques described herein are able to determine and remove redundant, unimportant, or incorrect rules and/or data, the system may make determinations as to improved rules and/or variables agnostic to the inputs, whether the input rules are accurate, and/or the type of data the system receives. For example, a model created by the techniques described herein may receive numerical data, binary data, ordinal data, continuous data, categorical data, or any other similar data type as input and use the distinct types in conjunction. As such, the system can manage noise that would otherwise destroy any convergence that standard techniques could potentially attain.

The techniques described herein relate to implementing machine-learning-based prediction systems that generate clinically relevant classifications, trajectories, and recommendations for patient outcomes associated with cerebrovascular accidents (strokes). More particularly, the disclosed techniques relate to utilizing medical imaging data (e.g., MRI, CT, or CTA data) and contextual information derived from electronic health records (EHR) to predict stroke outcomes and generate corresponding treatment recommendations. In some embodiments, the systems of the present invention include one or more models developed using and/or including tropical geometry-based fuzzy neural networks (TGFNNs), allowing interpretable, transparent, and clinically actionable stroke outcome predictions and recommendations.

Additionally, the disclosed techniques described herein provide further technical improvements by integrating embedding vectors derived from medical imaging data, generated using specialized embedding methods such as deep autoencoders. By reducing the dimensionality and extracting meaningful latent features from high-dimensional imaging data and EHR-derived clinical variables, the disclosed methods achieve improved predictive performance and computational efficiency (e.g., relative to generating outcome predictions directly from raw imaging and clinical datasets). In some embodiments, the invention described herein utilizes a learning with partially available privileged information (LUPAPI) framework that leverages complete medical datasets (e.g., including variables, or outcomes, not available for new patients at the time of prediction) enabling models to robustly generalize and remain operationally effective, even when patient information is incomplete or unavailable at decision-making time. These aspects combine to provide significant improvements in predictive modeling accuracy, computational efficiency, and clinical decision support capabilities not available using conventional outcome prediction methods.

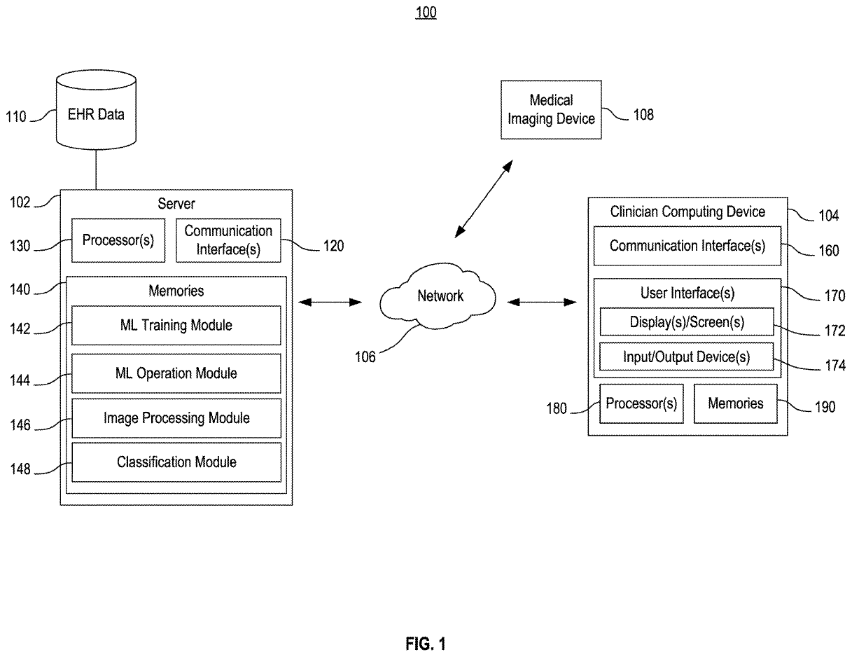

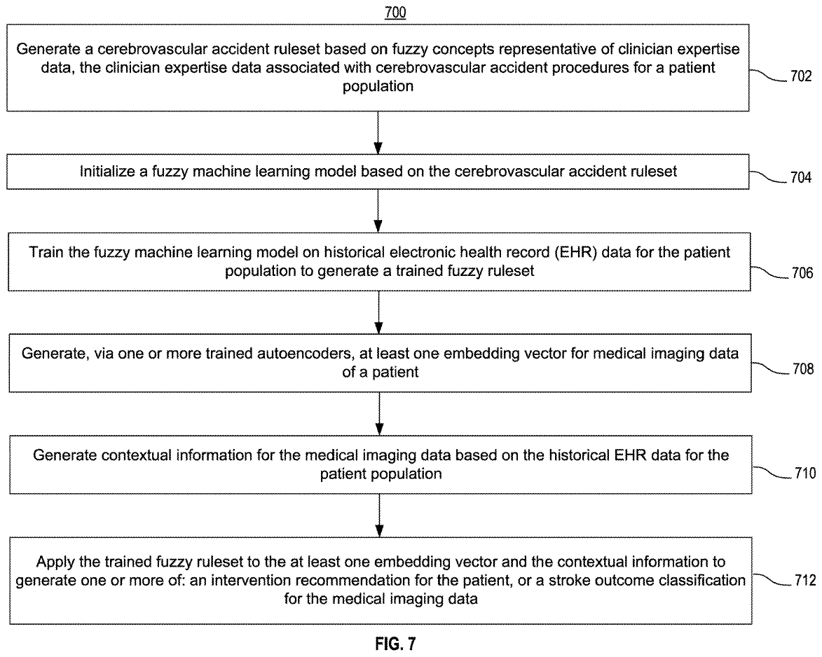

Referring now to the drawings, FIG. 1 is a block diagram representative of an example computing environment 100 capable of implementing the example methods and/or operations described herein, including, for example, one or more steps of the computer-implemented method 700 of FIG. 7 discussed in greater detail below. In some embodiments, the computing environment may be or may be associated with a medical environment. The computing environment 100 of FIG. 1 includes a server computing device 102, a clinician computing device 104, and one or more networks 106. In some embodiments, the computing environment 100 further includes one or more medical imaging devices 108. Additionally, or alternatively, the computing environment 100 may further include an electronic health record (EHR) database 110 and/or another database storing information for a patient population. The exemplary network 106 of FIG. 1 may be a single communication link directly connecting the server computing device 102 and the clinician computing device 104 (e.g., a direct wireless link), or one or more networks 106 may include multiple links (e.g., connecting the server computing device 102, the clinician computing device 104, the medical imaging device 108, the EHR database 110, and/or an additional computing device) and/or communication networks of one or more types (e.g., one or more wired and/or wireless local area networks (LANs), and/or one or more wired and/or wireless wide area networks (WANs) such as the Internet, public networks, private networks, etc.). The network 106 may include any number of network devices, such as gateways, switches, routers, modems, repeaters, and wireless access points, among others. Additionally, or alternatively, the network 106 may also include computing devices such as computer servers. For ease of reading herein (and not for limitation purposes), the one or more networks 106 may be referred to using the singular tense.

The server computing device 102 may be an individual server, a group (e.g., cluster) of multiple servers, a computing device (e.g., a personal computer, a laptop, a smart phone, a tablet, a wearable device, etc.), or another suitable type of computing device or system (e.g., a collection of computing resources). The server computing device 102 may include one or more communication interfaces 120, one or more processors 130, and one or more memories 140. Further, the memories 140 may have stored thereon one or more modules (e.g., one or more sets of instructions) including a machine learning (ML) training module 142, an ML operation module 144, an image processing module 146, a classification module 148.

The one or more communication interfaces 120 may enable communication with other machines (e.g., components of the computing environment 100 such as clinician computing device 104, medical imaging device 108, EHR database 110, etc.) via, for example, the one or more networks 106. The example communication interface 120 may include any suitable type of communication interface(s) (e.g., wired and/or wireless interfaces) configured to operate in accordance with any suitable protocol(s). For example, the communication interfaces 120 may be configured to transmit and receive data using a suitable wired communication protocol such as an Ethernet protocol, a USB protocol, a UART protocol, an I2C protocol, a SPI protocol, or wireless communication protocols such as a Bluetooth protocol, a Wi-Fi® (IEEE 802.11 standard) protocol, a near-field communication (NFC) protocol, a cellular (e.g., GSM, CDMA, LTE, WiMAX, etc.) protocol, a peer-to-peer wireless protocol, a short-range wireless protocol, and/or other suitable wired or wireless communication protocols. In some embodiments, the communication interface 120 may utilize a combination of such protocols to improve data throughput and/or efficiency. In some embodiments, the communication interface 120 may be a network interface controller (NIC) and may include any suitable NIC, such as wired/wireless controllers (e.g., Ethernet controllers), and facilitate bidirectional/multiplexed networking over the network 106 between the server computing device 102 and the clinician computing device 104 and/or other components of the environment 100 (e.g., medical imaging device 108, EHR database 110, etc.). In some embodiments, the communication interface 120 may include advanced features such as hardware acceleration, specialized networking protocols, etc.

The processors 130 may include one or more microprocessors, controllers, and/or any suitable type of processor, and the memories 140 (e.g., volatile memory, non-volatile memory, non-transitory computer-readable media, etc.) may be accessible by the processor 130 (e.g., via a memory controller). For example, the one or more processors 130 may include one or more central processing units, one or more graphics processing units, one or more field-programmable gate arrays, one or more application-specific integrated circuits, one or more tensor processing units, one or more digital signal processors, one or more neural processing units, one or more RISC-V processors, one or more coprocessors, one or more specialized processors/accelerators for artificial intelligence or machine learning-specific applications, one or more microcontrollers, etc. The processor 130 may interact with the memory 140 to obtain, for example, machine-readable instructions and/or computer-executable instructions stored in the memory 140 corresponding to, for example, the operations represented by the flowcharts of this disclosure (e.g., the computer-implemented method 700 of FIG. 7). For example, the memories 140 may include one or more random access memories, one or more read-only memories, one or more cache memories, one or more hard disk drives, one or more solid-state drives, one or more non-volatile memory express, one or more optical drives, one or more universal serial bus flash drives, one or more external hard drives, one or more network-attached storage devices, one or more cloud storage instances, one or more tape drives, etc. Additionally, the processors 130 may be communicatively coupled to and/or control the communication interface 120 to transmit and/or receive various information pursuant to executing of the instructions stored on the memories 140 via, for example, the network 106.

As noted above, the memories 140 may have stored thereon one or more modules, for example, as one or more sets of computer-executable instructions. Further, the memories 140 may include an ML training module 142, an ML operation module 144, an image processing module 146, a classification module 148. In some aspects, the memories 140 may have stored thereon one or more additional modules (not depicted). For example, the memories 140 may include additional storage, such as one or more operating systems (e.g., Microsoft Windows, GNU/Linux, Mac OSX, etc.). The operating systems may be configured to run the modules stored on the memories 140 (e.g., modules 142, 144, 146, 148) during operation of the server computing device 102. The modules stored on the memories 140 may be implemented using any suitable computer programming language(s) (e.g., Python, JavaScript, C, C++, Rust, C#, Swift, Java, Go, LISP, Ruby, Fortran, etc.). Further, the modules stored on the memories 140 may be configured to communicate with one another (e.g., via inter-process communication, via a bus, via message queues, etc.). In some embodiments, the modules stored on the memories 140 may respond to network requests (e.g., via an application programming interface) or other requests received via the network 106 (e.g., via the clinician computing device 104 or other components of the environment 100). In some implementations, the memories 140 may store data structures and/or information related to, for example, the modules (e.g., modules 142, 144, 146, 148) and/or algorithms used in training, testing, or utilizing models to create, extract, and/or improve rulesets according to analyzed input data and/or rulesets as described in more detail below.

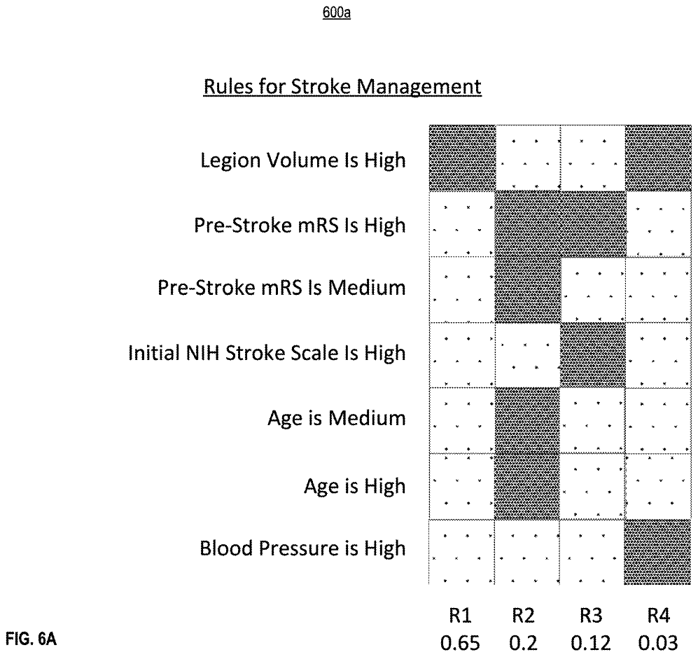

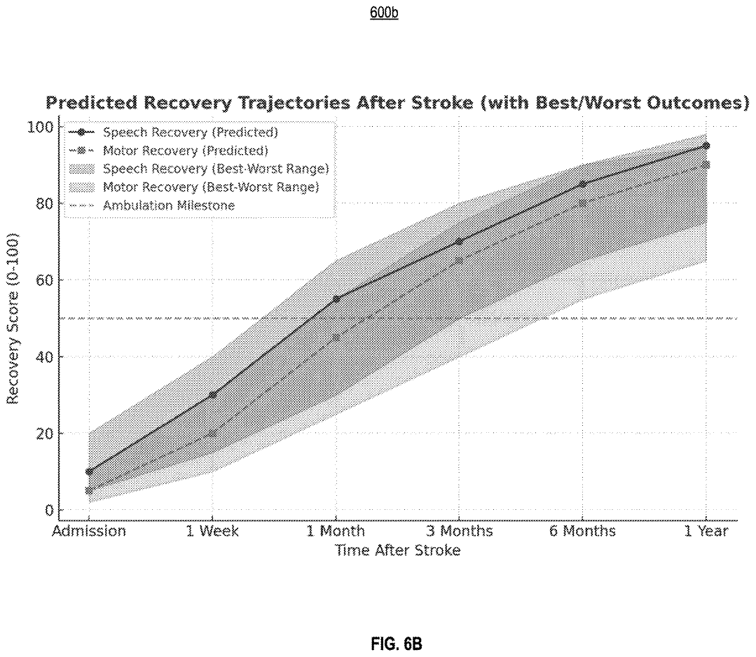

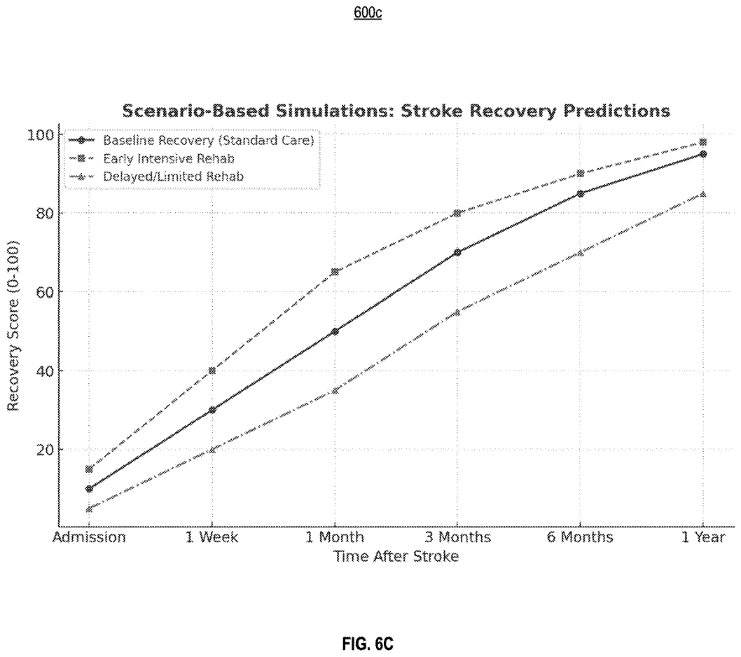

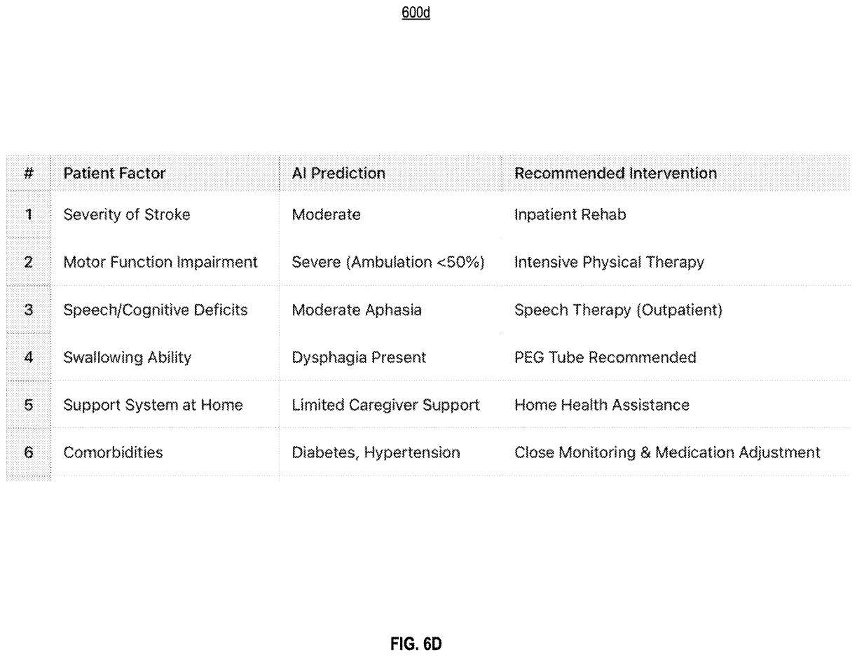

The server computing device 102 may also include one or more I/O devices (not depicted) and/or one or more displays/screens (not depicted), e.g., like the displays/screens 172 and the I/O devices 174 of the clinician computing device 104. The one or more I/O devices and/or the one or more displays/screens of the server computing device 102 may enable receipt of user input and communication of output data to the user. For example, the I/O devices and/or the displays/screens may present information in connection with the execution of instructions stored on the memories 140 for a user to view and/or otherwise perceive (e.g., the graphical user interfaces depicted in FIGS. 6A-6D). Similarly, the I/O devices and/or the displays/screens of the server computing device 102 may receive manual adjustments, selections, and/or data, and/or may allow a user to interact in any of a variety of manners with the processors 130 during execution of the instructions stored on the memories 140.

In some embodiments, a user may review data from, and/or provide data to, the modules of the memory 140 via the I/O devices of the server computing device 102 (not depicted) and/or the input/output (I/O) devices 174 of the clinician computing device 104. For example, the server computing device 102 may send data and/or information from modules 142, 144, 146, 148 to the computing device 104 to be presented via the user interface 170, and the server computing device 102 may receive inputs provided by a user (e.g., input data for modifying, adjusting, or otherwise interacting with a classification, model, ruleset, etc.) via the user interface 170 from the computing device 104. In some embodiments, the server computing device 102 may send data and/or information from the modules 142, 144, 146, 148, to various components of the computing environment 100 (e.g., clinician computing devices 104, medical imaging devices 108, EHR database 110, and/or one or more additional computing devices). For example, the server computing device 102 may send data and/or information from the ML operation module 144 and/or the classification module 148 to one or more databases/datastores of the computing environment 100 (e.g., EHR database 110) for subsequent storage.

The clinician computing device 104 includes one or more communication interfaces 160, one or more user interfaces 170, one or more processors 180, and one or more memories 190. In some embodiments, the clinician computing device 104 may be included in and/or associated with a medical environment (e.g., like server computer device 102). The clinician computing device 104 may be a computing device (e.g., a personal computer, a laptop, a smart phone, a tablet, a wearable device, etc.), or another suitable type of computing device or system (e.g., a collection of computing resources). In some embodiments, one or more clinician computing devices 104 may be included in the computing environment 100—for example, a user may access a first clinician computing device 104 that is a personal computer, a user may access a second clinician computing device 104 that is a smart phone, etc. A user of the clinician computing device 104 may be a clinician (e.g., a doctor, a physician, a surgeon, etc.) or another medical or healthcare professional (e.g., a nurse, a physician assistant, etc.). For ease of reading (and not limitation) purposes, the one or more clinician computing devices 104 may be referred to herein using the singular tense.

The one or more communication interfaces 160 may, like the communication interface 120, enable communication with other devices (e.g., server computing device 102) via, for example, the one or more networks 106. The example communication interface 160 may include any suitable type of communication interface(s) (e.g., wired and/or wireless interfaces such as a network interface controller) configured to operate in accordance with one or more suitable protocol(s). In some embodiments, the communication interface 160 may be a network interface controller (NIC) and may include any suitable NIC, such as wired/wireless controllers (e.g., Ethernet controllers), and facilitate bidirectional/multiplexed networking over the network 106 between the clinician computing device 104 and the server computing device 102 and/or other components of the environment 100 (e.g., medical imaging device 108, EHR database 110, another computing device, etc.). In some embodiments, the communication interface 160 may include advanced features such as hardware acceleration, specialized networking protocols, etc.

The one or more user interfaces 170 of the clinician computing device 104 may receive user input and communicate output data to the user. Further, the user interface 170 may be configured to present interactive graphical user interfaces (GUIs) to a user and receive feedback data from the user (e.g., via the input/output devices 174). For example, the user interface 170 may present interactive graphical user interfaces and/or graphical representations for a generated classification of a medical image, or imaging data, to a user (e.g., a clinician), such as the graphical user interfaces depicted in FIGS. 6A-6D, as discussed in greater detail below. As another example, a clinician may provide feedback data, such as an adjusted classification or an adjustment to a rule of a trained ruleset, using the user interface 170. Continuing with this example, the input data from the user interface 170, from a clinician, may be sent to the server computing device 102 and the ML operation module 144 may update, change, modify, and/or retrain the model or ruleset based on the feedback/input data.

The user interfaces 170 may include one or more input/output (I/O) device(s) 174. For instance, the I/O devices 174 may include one or more suitable types of user input devices, such as keyboards, touch screen displays, microphones, mice, touchpads, and/or any suitable types of remote and/or local user input devices. Similarly, the I/O devices 174 may include one or suitable types of output devices, such as touch screen displays, speakers, and the like. In some embodiments, the I/O devices 174 may include one or more display(s)/screen(s) 172 for presenting or displaying information to a user. The one or more displays/screens 172 may use any suitable display technology (e.g., LED, OLED, LCD, etc.). For example, the I/O devices 174 and/or the displays/screens 172 may be configured to present graphical representations of information and/or data associated with the embodiments described herein, such as information related to a trained model (e.g., a ML classifier, an autoencoder, a fuzzy neural network, etc.) or ruleset, information relating to one or more particular rules of a ruleset, information related to training a model or a ruleset (e.g., information and/or data from the ML operation module 144). In some embodiments, the I/O device 174 and the display/screen 172 are integrated as a touchscreen display. In some embodiments, the user interfaces 170, the I/O devices 174, and/or the displays/screens 172 are not integral to the clinician computing device 104 and receive instructions from the clinician computing device 104 via wired and/or wireless transmissions over communication interface 160, for example. In some embodiments, the user interfaces 170, the I/O devices 174, and/or the displays/screens 172 may include one or more local interfaces, and/or may include one or more remote interfaces that are communicatively connected to the clinician computing device 104 via the network 106 (e.g., that are provided by an application, web browser, or other software executing on a device of a user). For ease of reading (and not limitation) purposes, the user interfaces 170, the I/O devices 174, and/or the displays/screens 172 may be referred to herein using the singular tense.

The processors 180 may include one or more microprocessors, controllers, and/or any suitable type of processor (e.g., one or more processors similar to the processors 130), and the memories 190 (e.g., one or more volatile memories, one or more non-volatile memories, or one or more non-transitory computer-readable media similar to the memories 140) may be accessible by the processor 180 (e.g., via a memory controller). The processor 180 may interact with the memory 190 to obtain, for example, machine-readable instructions and/or computer-executable instructions stored in the memory 190 corresponding to, for example, one or more of the operations represented by the flowcharts of this disclosure (e.g., the method 700 of FIG. 7). Additionally, the processors 180 may be communicatively coupled to and/or control the communication interface 160 to transmit and/or receive various information pursuant to executing of the instructions stored on the memories 190 and/or the memories 140 via, for example, the network 106.

As mentioned above, the memories 190 may have stored thereon one or more modules, for example, as one or more sets of computer-executable instructions. In some aspects, the modules may include additional storage, such as one or more operating systems (e.g., Microsoft Windows, GNU/Linux, Mac OSX, etc.), like, for example, memories 140. The operating systems may be configured to run the modules during operation of the clinician computing device 104. For example, the modules may include additional modules and/or services for receiving and processing data from one or more other components of the environment 100 such as the server computing device 102, the medical imaging device 108, or the EHR database 110. The modules may be implemented using any suitable computer programming language(s) (e.g., Python, JavaScript, C, C++, Rust, C#, Swift, Java, Go, LISP, Ruby, Fortran, etc.). The modules may be configured to communicate with one another (e.g., via inter-process communication, via a bus, via sockets, pipes, message queues, etc.). In some embodiments, the modules may respond to network requests or other requests received via the network 106 (e.g., via the server computing device 102 or other components of the environment 100).

The medical imaging devices 108 of the computing environment may be a medical imaging device capable and/or configured to capture imaging data of patients, or a particular body part of patients. For example, the medical imaging device 108 may be a magnetic resonance imaging (MRI) machine (e.g.,), a computed tomography (CT) machine (e.g.,), a computed tomography angiography (CTA) machine (e.g.,), or another type of medical imaging device (e.g., an X-ray machine). In some embodiments, the medical imaging device(s) 108 receives instructions from the server computing device 102 and/or the clinician computing device 104 via wired and/or wireless transmissions over communication interface 120 and/or communication interface 160 (e.g., and/or a communication interface of the medical imaging device 108), for example. The medical imaging device 108 may transmit medical imaging data (e.g., MRI data, CT imaging data, CTA imaging data, etc.) to the server computing device 102 and/or the clinician computing device 104. For ease of reading (and not limitation) purposes, the one or more medical imaging devices 108 may be referred to herein using the singular tense.

The electronic health record (EHR) database 110 of the computing environment 100 may store historical medical data associated with one or more populations, such as EHR data for one or more patient populations. For example, the patient population database 114 may store historical medical data associated with a population of patients who have experienced a cerebrovascular accident (a stroke). The historical medical data for a population stored on the EHR database 110 may include electronic health records (EHR) for a plurality of patients in the population and/or historical clinical data (e.g., procedural data, clinical guidelines, clinician notes, etc.) for a plurality of patients in the population. For example, the EHR data may include medical history data (e.g., past illnesses, past surgeries, family medical history, etc.), medication data, biographic information (e.g., gender, height, weight, age), medical conditions, etc. As another example, the historical clinical data may include vital signs for a patient over a period of time (e.g., vital signs data for the duration of a patient's recovery from a surgery, other vital signs data collected for a patient over a period of time, routine check in/up data, and/or other clinical data collected over other periods of time). In some embodiments, the EHR database 110 may store data in one or more structured formats. For example, the EHR database 110 may store various sets of training data (e.g., within one or more vector data stores or caches) for the example machine learning models described herein.

In some embodiments, the EHR database 110 and/or another datastore (not explicitly depicted) of the computing environment 100 (e.g., a module stored on the memories 140 such as classification module 148, clinician computing device 104, an external datastore, etc.) includes one or more untrained stroke management rulesets and one or more trained stroke management rulesets. Typically, an example patient may be associated with one or more populations of patients (e.g., the patients that have experienced more than one stroke, the patients of a particular demographic, etc.), and further, stroke management procedures administered by clinicians may differ between populations of patients. In some embodiments, the ML operation module 144 and/or the classification module 148 may generate untrained stroke management rulesets by encoding clinician expertise data associated with a stroke management procedure for a population into fuzzy concepts. In some embodiments, the classification module 148 may generate trained stroke management rulesets by training untrained rulesets on, for example, historical clinical data. Generally speaking, the trained rulesets may encode stroke management procedure(s) specific to a population of patients and the classification module 148 may use the trained rulesets to evaluate or recommend stroke management for a patient of the corresponding population of patients.

The EHR database 110 and/or the additional datastore of the computing environment 100 can include servers or other computing devices to provide input data and/or input rules for the classification module 148. The input data and/or input rules can include raw data, such as cerebral blood flow (CBF) measurements; processed data, such as survey data; fuzzy rules, such as a rule stating that if infarct is large, and patient has atrial fibrillation (AF), then evaluate for ACT (anticoagulation therapy) initiation; or any other similarly desired input data. In further implementations, the EHR database 110 and/or the additional datastore of the computing environment 100 can receive and store data such as improved and/or new rulesets from the classification module 148. As mentioned above, a patient may be associated with one or more populations of patients. In some embodiments, the additional datastore and/or the EHR database 110 may store clinician expertise data associated with stroke management procedures for a population of patients. In some embodiments, the additional datastore and/or the EHR database 110 may store untrained and/or trained stroke management rulesets generated based on clinician expertise data associated with stroke management procedure for a population of patients including the patient.

Returning to the server computing device 102, the machine learning (ML) training module 142 stored on the memories 140 may include instructions for operating and/or training one or more machine learning models (e.g., an autoencoder, a machine learning classifier, a fuzzy machine learning model, etc.) and/or instructions for performing one or more of the operations described herein with respect to generating classifications of medical imaging data and/or trained fuzzy rulesets. In some embodiments, the ML operation module 144 and/or the classification module 148 may generate stroke outcome classifications for a patient based on medical imaging data associated with the patient using one or more example machine learning models (e.g., trained via the ML training application 142). Typically, an example patient may be associated with one or more populations of patients (e.g., the patient has had a cerebrovascular accident, and the population consists of patients that have had cerebrovascular accidents). Further, the ML training module 142 may include instructions for training one or more machine learning models on EHR data and/or other medical information for a patient population associated with a patient of interest. In some embodiments, the ML training module 142 may train one or more ML models on clinical data such as medical information and documentation associated with the patient population (e.g., procedural data, clinical guidelines, clinician notes, medical interventions, and associated outcomes, etc.). Additionally, the ML training module 142 may train one or more ML models on medical imaging data associated with a patient population. For example, the ML training module 142 may train one or more ML models on medical imaging data of a particular modality (e.g., MRI data, CT imaging data, etc.) and/or on medical imaging data of varying modality (e.g., a multi-modal ML model). In some embodiments, the ML training module 142 may train one or more ML models on medical imaging data, and/or corresponding embedding vectors, and contextual information associated with the medical imaging data (e.g., generated via one or more other trained ML models).

For example, the ML training module 142 may include instructions for training one or more autoencoders to generate embedding vectors for medical imaging data. An example autoencoder, trained via ML training module 142, may compress medical imaging data into lower dimensional embedding vectors. Furthermore, the ML training module 142 may be an autoencoder on historical medical images associated with a particular patient population (e.g., a population associated with cerebrovascular accidents). Additionally, or alternatively, the ML training module 142 may include instructions for training one or more autoencoders on historical EHR data for a patient population of interest to generate contextual information for medical imaging data.

As another example, the ML training module 142 may include instructions for training one or more ML classifier models on embedding vectors and contextual information, from one or more example autoencoders trained via ML training module 142, to generate classifications for the corresponding medical imaging data. In some embodiments, the ML training module 142 may generate an example ML classifier by training an artificial neural network (ANN) model to learn a function that maps a standard feature space of historical medical image embeddings and a privileged feature space of the historical EHR data to an outcome. Further, the ML training module 142 may train a model with information that is not available during an inference phase, sometimes referred to as learning with partially available privileged information (LUPAPI). For example, privileged information may include outcomes for historical patients associated with a particular population (e.g., the outcomes are known) and the ML training module 142 may provide such privileged information to a model during a training phase, whereas an outcome for a patient of interest associated with the population may be unknown (e.g., the patient is currently recovering, the outcome is ongoing/developing).

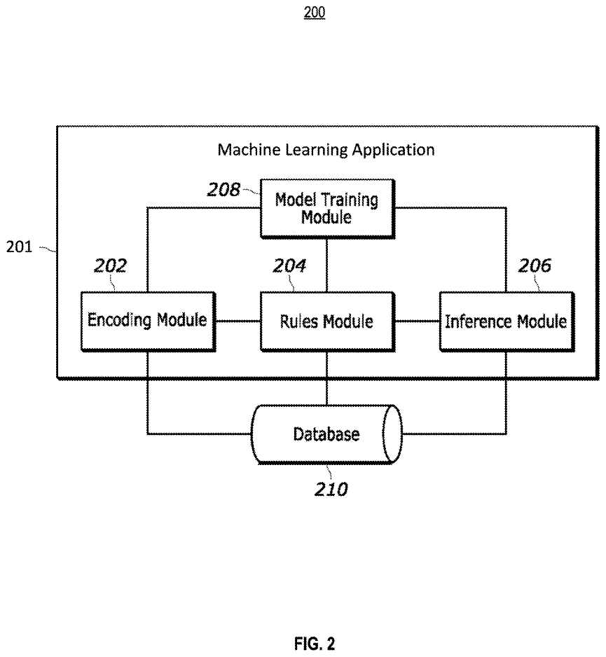

The ML operation module 144 stored on the memories 140 may include instructions for operating one or more example ML models described herein, such as one or more autoencoders, one or more ML classifiers, one or more trained fuzzy rulesets or fuzzy neural networks, etc. Generally, the ML operation module 144 may include instructions for applying one or more ML models (e.g., trained via ML training module 142) to medical imaging data associated with a patient that has experienced a cerebrovascular accident to generate one or more stroke outcome classifications for the patient, as described further below with respect to FIGS. 2-5.

For example, the ML operation module 144 may include instructions for generating one or more embedding vectors for medical imaging data associated with a cerebrovascular accident of a patient using one or more autoencoders. Further, the ML operation module 144 may apply an autoencoder, trained (e.g., via ML training module 142) on historical medical images for a patient population associated with a patient of interest, to medical imaging data for the patient of interest. In some embodiments, the ML operation module 144 receives/obtains multi-modal imaging data for a patient of interest (e.g., a patient that has had a cerebrovascular accident). Like the above, the ML operation module 144 may apply one or more autoencoders to medical imaging data for the patient of interest to generate one or more embedding vectors. For example, the ML operation module 144 may apply an individual multi-modal autoencoder (e.g., trained via ML training module 142 to process multi-modal imaging data) or multiple autoencoders (e.g., each trained via ML training module 142 to process a particular modality of imaging data) to such multi-modal imaging data.

As another example, the ML operation module 144 may include instructions for generating contextual information for medical imaging data associated with the cerebrovascular accident of the patient based on historical EHR data (e.g., from database 110) for the corresponding patient population using one or more autoencoders. Further, the ML operation module 144 may apply an autoencoder, trained (e.g., via ML training module 142) on historical EHR data for a corresponding patient population, to medical imaging data for a patient of interest. In some embodiments, the autoencoder may be further trained (e.g., via ML training module 142) on corresponding medical images for the patient population. Additionally or alternatively, the ML operation module 144 may apply an autoencoder (e.g., trained on historical EHR data and corresponding medical images) to medical imaging data and medical information (e.g., EHR data, clinician notes, vital signs data, active medical device data, etc.,) for a patient to generate the contextual information. In some embodiments, the contextual information may include relevant medical information identified (e.g., via one or more trained autoencoders) based on the medical imaging data for a patient, such as procedural data, clinical guidelines, clinician notes, medical interventions, and associated outcomes, etc.

Yet another example, the ML operation module 144 may include instructions for generating classifications of medical imaging data using one or more ML classifiers. Further, the ML operation module 144 may apply a ML classifier model, trained (e.g., via ML training module 142) on historical medical images and historical outcomes for patients associated with a particular population, to medical imaging data for a patient of interest and contextual information for the medical imaging data generated (e.g., via ML operation module 144) using one or more trained autoencoders. In some embodiments, the ML operation module 144 may generate classification for medical imaging data by applying one or more ML classifier models to the medical imaging data and EHR data for a patient. For example, the ML operation module 144 may obtain EHR data for the patient (e.g., from EHR database 110 or another medical information data source) and augment the contextual information with the EHR data for the patient.

The image processing module 146 stored on the memories 140 may include instructions for obtaining and processing medical imaging data from one or more components of the computing environment 100. For example, the image processing module 146 may obtain medical imaging data from the medical imaging device 108. In some embodiments, the image processing module 146 includes instructions for controlling the medical imaging device(s) 108 to capture medical imaging data and/or images for a patient. As another example, the image processing module 146 may obtain medical images from the EHR database 110, and/or another data store of the computing environment 100 storing historical medical images for a patient population. In some embodiments, the image processing module 146 may include instructions for processing medical imaging data (e.g., from EHR database 110, medical imaging device 108, another computing device). For example, the image processing module 146 may include instructions for mapping medical imaging data to a standardized coordinate system, such as the Montreal Neurological Institute (MNI) coordinate system. Additionally, or alternatively, the image processing module 146 may include instructions for normalizing medical imaging data by applying a brain mask (e.g., a grey matter mask, a white matter mask, a whole brain mask, etc.) to the medical imaging data and/or images. The image processing module 146 may include instructions for processing, standardizing, and/or normalizing multi-modal imaging data and/or imaging data of varying modalities. For example, the image processing module 146 may process MRI data, CT imaging data, CTA imaging data, etc., for input to one or more ML models (e.g., via ML operation module 144).

The classification module 148 stored in the memories 140 may include instructions for generating stroke outcomes classifications for medical imaging data associated with a patient. For example, the classification module 148 may include instructions for generating a length of stay (LOS) classification, a recovery timeline classification (e.g.: recovery of motor functions, recovery of speech functions, predicted time to ambulation), a modified Rankin scale (mRS) classification (e.g., a classification measuring the degree of disability or dependence in daily activity of a patient), a discharge classification (e.g., discharge to home, discharge to inpatient rehabilitation, discharge to nursing facility, etc.), an intervention classification (e.g., need for a tracheostomy, need for a PEG tube, etc.), a mortality risk classification, and/or a combined or aggregated classification (e.g., incorporating features and/or concepts from the other classifications). The classification module 148 may also include instructions for presenting or displaying the example classification described herein. For example, the classification module 148 may present and/or display a classification and/or information associated therewith via the user interface 170. Expanding on this example, the classification module 148 may present classifications in a graphical form, as visual risk scores, as recovery trajectory timelines, as scenario-based simulations, as described further below with respect to FIGS. 6A-6D. Additionally, the classification module 148 may present (e.g., via user interface 170) one or more stroke outcome classifications and the corresponding medical imaging data to facilitate review of the classification by a clinician or medical practitioner operating the clinician computing device 104. In some embodiments, the classification module 148 may provide the generated classification (e.g., from ML operation module 144) to one or more ML models, or generative AI models, to generate AI powered decision support information/context (e.g., a recommendation based on the generated classification, additional contextual information for the medical imaging data, etc.).

In some embodiments, the classification module 148 may generate various stroke outcome classification for a plurality of medical images associated with a patient and/or a cerebrovascular accident. As mentioned above, the ML operation module 144 may include instructions for obtaining EHR data associated with a patient to generate the stroke outcome classification for the corresponding medical imaging data. In some embodiments, the EHR data associated with the patient may include historical medical imaging data (e.g., historical MRIs, CT scans, etc.) for the patient of interest and the classification module 148 may generate (e.g., via ML operation module 144) one or more additional embedding vectors for the historical medical imaging data to be provided to the ML classifier model when tasked with generating a stroke outcome classification for the corresponding medical imaging data. Moreover, the classification module 148 may generate a stroke outcome classification for medical imaging data associated with a cerebrovascular accident of a patient based on historical medical images for the patient, thereby providing additional context to the ML classifier that may improve the accuracy and granularity of the generated stroke outcome classification.

In some embodiments, the medical imaging data obtained by the image processing module 146 (e.g., from the medical imaging device 108, from the EHR database 110, etc.) is multi-modal imaging data. Further, the classification module 148 may generate two or more embedding vectors for the multi-modal imaging data using at least two trained autoencoders (e.g., trained as described above with respect to the ML training module 142), with each vector of the two or more embedding vectors corresponding to a respective modality of the multi-modal imaging data. Additionally, the classification module 148 may generate a multi-modal stroke outcome classification of the medical imaging data via an ML classifier model (e.g., trained as described above with respect to the ML training module 142) and using the two or more embedding vectors and the contextual information for the medical imaging data.

In some embodiments, the medical imaging data obtained by the image processing module 146 includes a time series of medical images (e.g., medical images for a patient captured over a period, such as hours, days, weeks, months, etc.). Further, the classification module 148 may generate, via one or more trained autoencoders, one or more additional embedding vectors (e.g., in addition to the an embedding vector for the initial, or most recent, medical image or imaging data) for one or more additional medical images in the time series of medical images, with each additional embedding vector corresponding to a respective medical image of the time series of medical images. Additionally, the classification module 148 may generate a time-series stroke outcome classification of the medical imaging data using the additional embedding vectors, and the embedding vector for the initial medical imaging data and the corresponding contextual information.

In some embodiments, the classification module 148 may include instructions for generating, via a trained ML model (e.g., a trained ML classifier, a trained FNN, a trained fuzzy ruleset, etc.), a treatment recommendation for medical imaging data associated with a patient of interest and/or a cerebrovascular accident for the patient. For example, the classification module 148 may generate, via a trained ML model, a treatment recommendation based on the at least one embedding vector for the medical imaging data, the contextual information for the medical imaging data, and/or the stroke outcome classification for the medical imaging data. Expanding this example, the ML training module 142 may generate a fuzzy ruleset based on clinician expertise data for stroke management procedure. Further, the ML training module 142 may train the fuzzy ruleset on historical medical imaging data and associated contextual information to generate a trained fuzzy ruleset. In some embodiments, the classification module 148 may apply the trained fuzzy ruleset to the medical imaging data and contextual information for the patient to generate a treatment recommendation based on the ruleset (e.g., if X and/or Y, then Z).

In some embodiments, the image processing module 146 may receive and process volumetric (3D) medical imaging data or datasets. Further, conventional image-based clinical decision support systems rely on specific 2D slices or limited anatomical cross-sections, potentially missing clinically relevant spatial relationships or broader anatomical context within the complete 3D volume. In contrast, the techniques described herein include utilizing trained autoencoders to efficiently process entire volumetric images, thereby capturing richer and more comprehensive latent feature representations of complex brain anatomy, for example. Further, the ML training module 142 may train an autoencoder on historical volumetric imaging data to generate embedding vectors that capture clinically meaningful patterns across the entire volumetric context. Using the conventional techniques, employing comprehensive volumetric imaging data in this manner requires large and computationally intensive model architectures, making training more resource demanding as compared to conventional 2D approaches. However, the modular nature of the example autoencoders described herein substantially counteracts these challenges by enabling a single initial training step. Specifically, an example autoencoder (e.g., autoencoders, 504, 506) may be trained once on representative historical volumetric imaging data (e.g., volumetric imaging data representative of a patient population) to generate embedding vectors that can be reused multiple times across diverse downstream prediction tasks without requiring additional re-training. Thus, the disclosed embodiments may, while leveraging complete volumetric imaging information, reduce overall computational complexity and improve predictive performance.

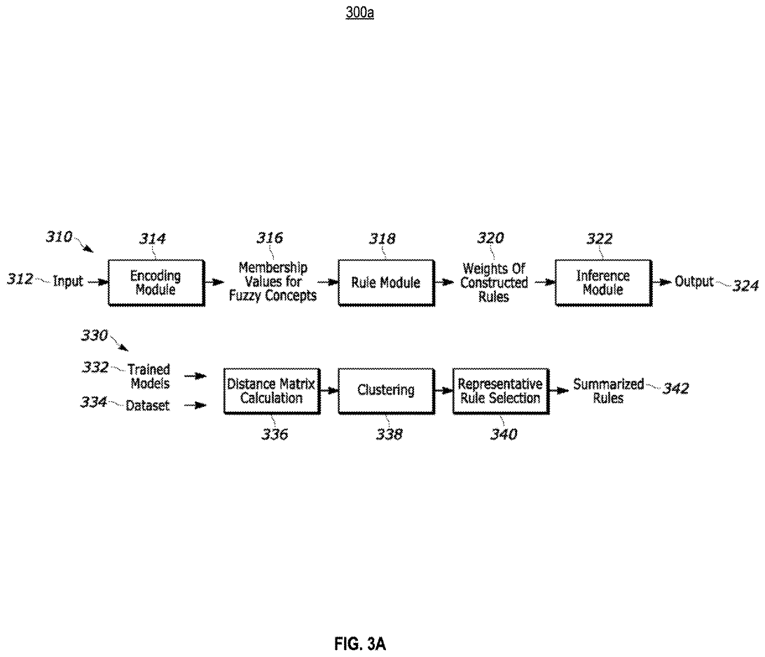

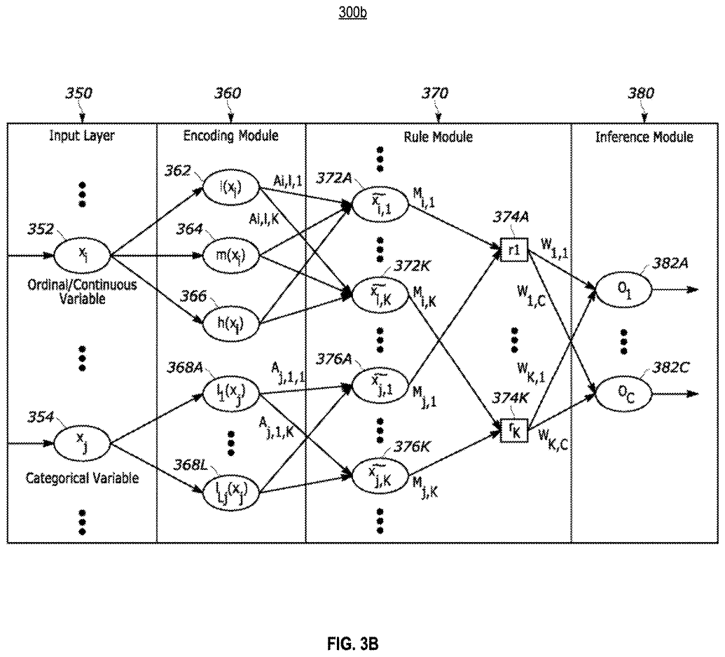

The classification module 148 may include instructions for implementing a fuzzy neural network architecture developed using tropical geometry, as described below with respect to FIGS. 2-3B. The classification module 148 provides an interpretable and transparent framework that encodes complex interactions between various clinical factors, medical imaging features, and related electronic health record (EHR) information into computationally efficient and human-understandable fuzzy rules. In some embodiments, the classification module 148 may be an application (e.g., machine learning application 201 of FIG. 2) comprising or including various modules and/or algorithms, such as encoding module 202, rules module 204, inference module 206, and/or model training module 208 of FIG. 2.

In some embodiments, the classification module 148 may include instructions for encoding clinician expertise data (e.g., clinician feedback data associated with stroke outcome prediction and treatment decisions) into fuzzy concepts (e.g., expressed via linguistic variables, such as “low severity,” “high risk,” “moderate impairment”). For example, clinician expertise data may include protocol-based and heuristic knowledge linking certain stroke characteristics observed in clinical practice (e.g., imaging findings, clinical conditions, or in-hospital interventions) to outcomes (e.g., length of stay, functional deficits, interventions needed). A fuzzy concept may have a corresponding trainable membership function that maps input data variables into numerical membership values between 0 and 1, thereby quantifying the degree of belief that the input data satisfies the corresponding fuzzy concept. Additionally, the classification module 148 may generate one or more fuzzy rulesets based on the encoded clinician expertise data and corresponding fuzzy concepts. Each fuzzy rule in an example ruleset may relate one or more fuzzy concepts (e.g., representing clinical heuristics or protocol-based knowledge) to predicted stroke outcomes or recommended treatments. For example, a fuzzy rule may be: “IF . . . THEN”.

Further, the classification module 148 may include instructions for training one or more fuzzy neural network models to refine fuzzy rulesets using a tropical geometry-based computational framework. For example, the classification module 148 may include instructions for initializing a neural network model based on a fuzzy ruleset generated based on clinician expertise data and subsequently training the initialized model on relevant historical datasets (e.g., historical medical imaging data, historical EHR information, and clinical outcome data from large patient population cohorts, such as patient populations associated with cerebrovascular accidents). An example of a tropical geometry-based computational framework may include approximating continuous numerical representations of originally piecewise-defined fuzzy rules. Furthermore, the tropical geometry-based fuzzy neural network (TGFNN) techniques described herein enable efficient gradient-based optimization and learning within such a neural network (e.g., initialized using piecewise-defined fuzzy rules), facilitating the generation of a refined/tuned fuzzy rulesets that accurately encode complex clinical decision-making processes.

The classification module 148 may include instructions for applying a trained fuzzy ruleset to observed patient data (e.g., embedding vectors derived from medical imaging data, contextual information derived from historical EHR data, other clinician data/information, generated classifications, etc.) to generate transparent and clinically interpretable predictions or recommendations. By leveraging the tropical geometry-based fuzzy neural networks (TGFNNs), comprehensive and interpretable stroke outcome predictions and treatment recommendations may be generated from medical imaging embedding vectors, multi-source clinical data, and EHR-extracted contextual variables, enabling clinical validation and implementation without dependence on opaque “black-box” models or extensive data requirements.

In some embodiments, the transparency and interpretability of the TGFNN provided by the classification module 148 may facilitate manual adjustments of individual rules based on clinician feedback, changing clinical standards, or emerging clinical practices. For example, the classification module 148 may include instructions for presenting a graphical representation or visualization of a trained fuzzy rulesets and corresponding predictions or treatment recommendations via one or more graphical user interfaces (GUIs) (e.g., user interfaces 170 of clinician computing device 104). Further, the classification module 148 may include instructions for interpreting feedback data received via user interfaces 170 (e.g., feedback data from a clinician), or another user interface of the computing environment 100, and applying adjustments/updates to individual rules of a trained ruleset based on the feedback data.

In some embodiments, the classification module 148 may include instructions for integrating partially available privileged information during the model training process, such as retrospective outcome information or clinical knowledge not always immediately available at the point of care, further improving predictive accuracy and clinical relevance. For example, a GUI presenting a trained fuzzy ruleset and/or data associated therewith may visually depict relative rule importance and activation, enabling clinicians with intuitive mechanisms for validation, interaction, and fine-tuning.

In some embodiments, modules 142, 144, 146, 148 may include additional computer-executable instructions corresponding to, for example, one or more of the operations represented by the flowcharts of this disclosure (e.g., the computer-implemented method 700 of FIG. 7). For example, the ML operation module 144 and/or the classification module 148 may include instructions for generating a stroke outcome classification for medical imaging data corresponding to a cerebrovascular accident associated with a patient. Further, the modules stored on the memories 140 may include instructions for operating and applying (e.g., to medical imaging data and associated contextual information) various trained machine learning models, as discussed further below with respect to FIGS. 2-7.

Referring next to FIG. 2, an example application architecture 200 for a machine learning (ML) application 201 (e.g., stored on the memories 140 of the server computing device 102 discussed above with respect to FIG. 1) configured to execute various algorithms and/or modules to process data and train a machine learning model, such as encoding module 202, rules module 204, inference module 206, and/or model training module 208. In some embodiments, the components of the ML application 201 may be communicatively connected to a database 210 via, for example, the communication interface 120 of the server computing device 102. The ML application 201 may train and/or apply a model using a tropical geometry-based interpretable machine learning method. In some embodiments, the ML application 201 executes one or more additional modules such as the modules 142-148 of the memories 140.

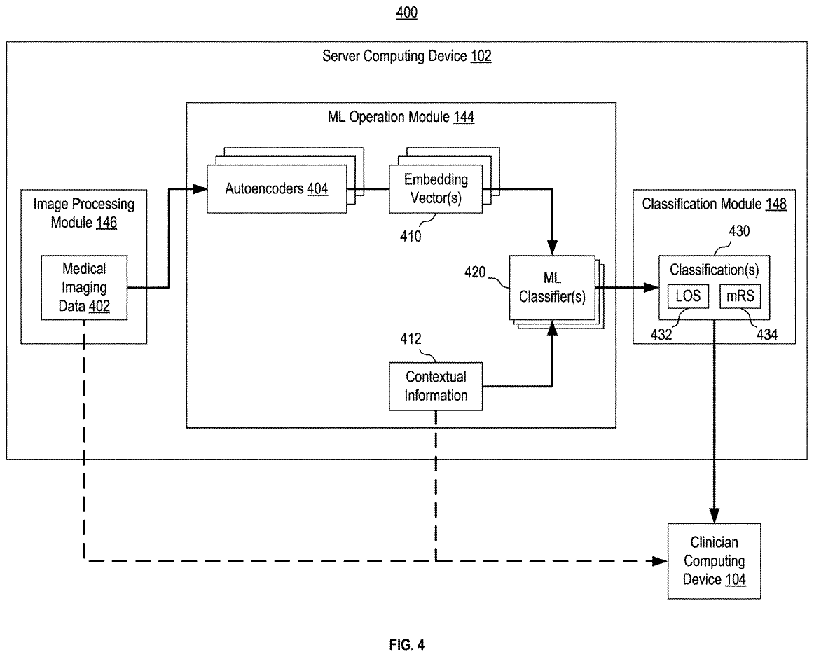

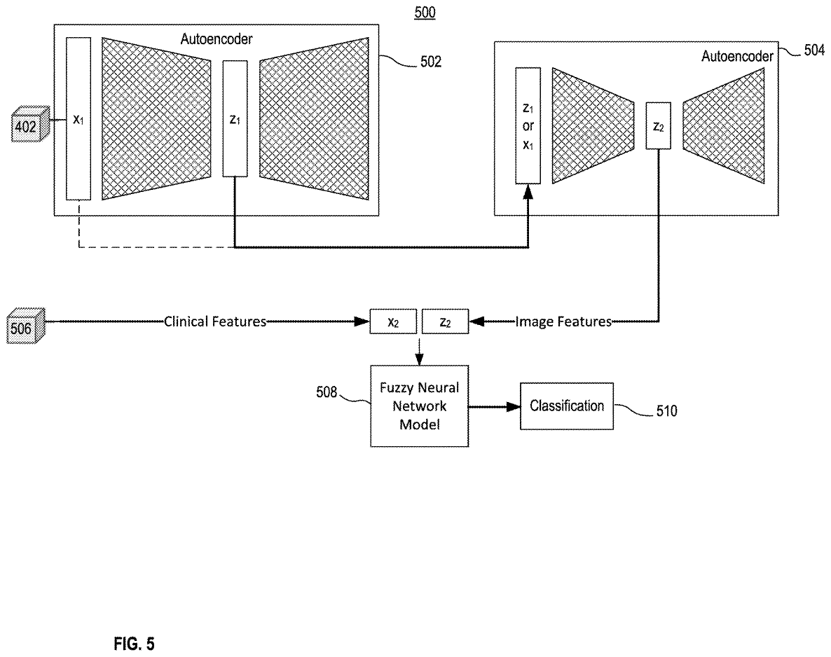

The ML application 201 may be capable of communicating with (e.g., via the communication interface 120 and over the network 106) components of the computing environment 100 and/or components of the application architecture 200, such as the one or more clinician computing devices 104, the EHR database 110, the medical imaging device(s) 108, one or more additional medical devices, and/or the database 210. Additionally, the ML application 201 may include one or more communication interfaces that facilitate information flow between the components of the ML application 201 (e.g., between the encoding module 202 and the rules module 204 and the inference module 206, between the model training module 208 and the encoding module 202 and/or the database 210, etc.). For example, input data (e.g., image embeddings and/or contextual embeddings, such as those described with respect to FIGS. 4 and 5 below) may be provided to an input layer of the module (not depicted) and passed sequentially to the encoding module 202, the rules module 204, and the inference module 206. In some embodiments, the modules 202, 204, 206 may be trained layers of an artificial neural network (e.g., a fuzzy neural network model) or another machine learning model.

The database 210 can maintain a data structure such as a table of virtual user identifiers, corresponding user identifiers and/or characteristics, and cookies associated with the virtual user identifiers. The database can further maintain one or more data structures regarding database or user device identifiers and/or information, such as a tree, a linked list, a table, a string, or a combination thereof. In some implementations, the database 210 is part of or is included in the memories 140.

As mentioned above, the ML application 201 may include several logic modules, such as the encoding module 202, the rules module 204, the inference module 206, and/or the model training module 208. Depending on the implementation, each of the encoding module 202, rules module 204, inference module 206, and/or model training module 208 can be implemented as a software module, hardware module, or a combination of both. For example, each module can include a processing unit, server, virtual server, circuit, engine, agent, appliance, or other logic device such as programmable logic arrays configured to communicate with the database 210 and/or with other components of the computing environment 100 via the network 106. The ML application 201 may include instructions which cause the ML application 201 to perform operations discussed below regarding any of and/or any combination of the encoding module 202, rules module 204, inference module 206, and/or model training module 208.

The encoding module 202 receives input data from the components of the computing environment 100. In particular, the encoding module 202 can receive input data from the database 210 and/or from any of the server computing device 102, the clinician computing device 104 (e.g., input data received via the input/output devices 174), the EHR database 110, the medical imaging device(s) 108, and/or one or more additional computing devices, via the network 106. The input data may be ordinal data, continuous data, categorical data, etc. Depending on the implementation, the input data can be raw data, such as cerebral blood flow (CBF) measurements; processed data, such as survey data; fuzzy rules, such as a rule stating that “if infarct is large (e.g., infarct volume is HIGH), and patient has atrial fibrillation (AF), then evaluate for ACT (anticoagulation therapy) initiation;” or any other similarly desired input data.

In some implementations, the encoding module 202 then uses fuzzy theory to encode variables into multiple fuzzy sets. The encoding module 202 assigns a membership value in the range of [0,1] to each variable based on the observed value for a given fuzzy set, indicating the confidence of the variable belonging to a given concept and/or set (i.e., with 0 referring to no confidence and 1 referring to complete confidence). In some implementations, the encoding module 202 uses membership functions to calculate the membership values. In further implementations, the model training module 208 trains the membership functions while training the machine learning model as a whole.

The encoding module 202 determines concepts for the input variables. In particular, the encoding module 202 encodes the variables into humanly understandable fuzzy concepts. The fuzzy concepts approximate the concepts used by human experts during decision-making. For example, an expert may describe a metric used in making a determination as “low,” “medium,” or “high” without having a firm definition of what constitutes each concept. As examples, a clinician may describe cerebral blood flow as low, medium, or high; a stockbroker may describe a stock as being low, medium, or high; and a network security analyst may describe a level of activity in a network as low, medium, or high. Each expert may make the determination without having a firm bound on what constitutes each category, and may even have crossover between categories (e.g., a heart rate is “a little high”).

The encoding module 202 then sets trainable membership functions for each of the concepts. For example, in some implementations, the encoding module 202 defines three functions, l(x), m(x), and h(x). In such implementations, the membership functions are defined as

$$l ( x ) = {f}_{{ϵ}_{1}} ( \frac{{a}_{i , 2} - x}{{a}_{i , 2} - {a}_{i , 1}} ) - {f}_{{ϵ}_{1}} ( \frac{{a}_{i , 1} - x}{{a}_{i , 2} - {a}_{i , 1}} ) , h ( x ) = {f}_{{ϵ}_{1}} ( \frac{x - {a}_{i , 3}}{{a}_{i , 4} - {a}_{i , 3}} ) - {f}_{{ϵ}_{1}} ( \frac{x - {a}_{i , 4}}{{a}_{i , 4} - {a}_{i , 3}} ) , and m ( x ) = {f}_{{ϵ}_{1}} ( \frac{x - {a}_{i , 1}}{{a}_{i , 2} - {a}_{i , 1}} ) - {f}_{{ϵ}_{1}} ( \frac{x - {a}_{i , 2}}{{a}_{i , 2} - {a}_{i , 1}} ) - {f}_{{ϵ}_{1}} ( \frac{{a}_{i , 3} - x}{{a}_{i , 4} - {a}_{i , 3}} ) + {f}_{{ϵ}_{1}} ( \frac{{a}_{i , 4} - x}{{a}_{i , 4} - {a}_{i , 3}} ) - 1 , where {f}_{{ϵ}_{1}} ( x ) = {ϵ}_{1} log ( 1 + exp ( \frac{x}{{ϵ}_{1}} ) ) ,$$

tunable hyperparameters a_{i,1}<a_{i,2}<a_{i,3}<a_{i,4}, and 0<∈1<1. In such implementations, a_{i,1}<a_{i,2}<a_{i,3}<a_{i,4} are trainable variables in the form of tunable hyperparameters. Depending on the implementation, the number of tunable hyperparameters varies depending upon the number of categories considered. Although four tunable hyperparameters are described above, it will be understood that any number of hyperparameters may be used. For example, the tunable hyperparameters may include a_{i,1}<a_{i,2}< ···<a_{i,w}<a_{i,w+1}. Similarly, the encoding module may calculate the membership functions according to the above using the tunable hyperparameters. Further, the membership functions have a smoothness modulated by ∈₁. Because

$${lim}_{{ϵ}_{1} \to 0} {f}_{{ϵ}_{1}} ( x ) = max ( 0 , x ) ,$$

when ∈₁ approaches 0, the membership functions approach trapezoidal or triangular membership functions.

As described above, the encoding module 202 then encodes the input variables xᵢ as membership values in the fuzzy concepts. It will be understood that though three concepts are described herein, the techniques as described herein may apply to any number of concepts, and three concepts are chosen for ease of illustration and explanation.

In some implementations, the encoding module 202 may determine whether to encode the variables into fuzzy concepts based on the type of data input received. For example, the encoding module 202 may determine to encode the input data variables into fuzzy concepts when the data input is an ordinal or continuous variable, but may instead directly encode categorical variables, such that xⱼ is encoded into l₁(xⱼ), l₂(xⱼ), . . . , l_{L}_{ⱼ}(xⱼ), where Lⱼ is the number of levels of xⱼ and only one of l₁(xⱼ), l₂(xⱼ), . . . , l_{L}_{ⱼ}(xⱼ) has a value of 1, while all others have a value of 0.

The rules module 204 determines the most relevant concept from each variable for each rule and calculates a firing strength (e.g., a weight) for each rule. In some implementations, the architecture of the rules module 204 includes two layers. In such implementations, the first layer of the rules module 204 selects the most relevant concept from each variable for each rule, and the second layer calculates the rule firing strength for each rule. Although the rules module 204 is described with respect to two layers, it will be understood that the rules module 204 may have fewer or more layers, depending on the implementation.

In some implementations, the rules module 204 determines the most relevant concept from each variable with respect to each rule using an attention matrix A. A is the partitioned matrix formed by concatenating submatrices A₁, A₂, . . . , A_{H}, where Aₕ is the attention submatrix for the input variable xₕ and H=I+J is the total number of input variables. Further, I and J are the total number of ordinal/continuous variables and categorical variables, respectively. In some implementations, the rules module 204 treats ordinal and continuous variables differently from categorical variables. For example, for an ordinal and/or continuous variable xᵢ, the submatrix Aᵢ with entries A_{i,m,n} has dimension Cₚ×K, where Cₚ is the number of concepts for ordinal or continuous variables, and K is the number of rules utilized in the network. In some implementations, Cₚ=3 and refers to the concepts “high,” “medium,” and “low” as described regarding the encoding module 202 above. Similarly, for a categorical variable xⱼ, the submatrix Aⱼ with entries A_{j,m,n} has dimension Lⱼ×K, where Lⱼ is the number of levels of xⱼ. As such, the attention matrix A has dimension (Cₚ·I+ΣⱼLⱼ)×K.

In implementations in which Cₚ=3, the entry A_{i,1,k} in the attention matrix may represent the contribution of the ordinal or continuous variable xᵢ being “low” to rule k, the entry A_{i,2,k} in the attention matrix may represent the contribution of xᵢ being “medium” to rule k, and the A_{i,3,k} in the attention matrix may represent the contribution xᵢ being “high” to rule k. Depending on the implementation, the number may vary, and any A_{i,n,k} may correspond with the appropriate concept. In further implementations, entries in the attention matrix are all trainable and constrained to [0,1] by an activation function, such as a hyperbolic tangent activation function. A higher value in A indicates a higher contribution. For an input variable xᵢ, the corresponding output from the rules module 204, or the first layer of the rules module 204 in implementations in which the rules module 204 includes multiple layers, is \tilde{x}ᵢ, a vector of length K. Further, \tilde{x}_{i,k} is the kth element of \tilde{x}ᵢ and represents the firing strength of xᵢ involved in the kth rule. In implementations in which the rules module 204 treats ordinal and continuous variables differently from categorical variables, then \tilde{x}_{i,k}=A_{i,1,k}l(xᵢ)+A_{i,2,k}m(xᵢ)+ . . . +A_{i,n,k}h(xᵢ) for an ordinal or continuous variable xᵢ, and

$${\tilde{x}}_{j , k} = {Σ}_{d = 1}^{{L}_{j}} {A}_{j , d , k} {l}_{d} ( {x}_{j} )$$

for a categorical variable xⱼ.

In some implementations, the rules module 204 calculates rule firing strength by a connection matrix M of dimension H×K. In further implementations, a second layer of the rules module 204 separate from the first layer of the rules module 204 that selects the most relevant concept from each variable with respect to each rule. The rules module 204 constructs the kth rule as a combination of \tilde{x}_{1,k}, . . . , \tilde{x}_{H,k}. An entry M_{i,k} in the connection matrix M denotes the contribution of xᵢ to the kth rule. In some implementations, entries in the connection matrix are all trainable and constrained to [0,1] by an activation function, such as a hyperbolic tangent activation function, and a higher value indicates a higher contribution.

In order to calculate rₖ, the firing strength of the kth rule, the rules module 204 defines a parameterized T-norm,

$${T}_{{ϵ}_{2}} ( x , y ) = {g}_{{ϵ}_{2}}^{- 1} ( {g}_{{ϵ}_{2}} ( x ) + {g}_{{ϵ}_{2}} ( y ) ) with {g}_{{ϵ}_{2}}^{- 1}$$

as the inverse of g_{∈}_{₂}. For 0<∈₂<1, g_{∈}_{₂} in the range of [0, ∞) is defined as

$${g}_{{ϵ}_{2}} ( x ) = ( \frac{{ϵ}_{2}}{1 - {ϵ}_{2}} ) ( 1 - {x}^{\frac{{ϵ}_{2} - 1}{{ϵ}_{2}}} ) and {g}_{{ϵ}_{2}}^{- 1}$$

is defined as

$${g}_{{ϵ}_{2}}^{- 1} ( z ) = {( 1 - \frac{1 - {ϵ}_{2}}{{ϵ}_{2}} z )}^{\frac{{ϵ}_{2}}{{ϵ}_{2} - 1}} . Therefore , {T}_{{ϵ}_{2}} ( x , y ) = {( {x}^{\frac{{ϵ}_{2} - 1}{{ϵ}_{2}}} + {y}^{\frac{{ϵ}_{2} - 1}{{ϵ}_{2}}} - 1 )}^{\frac{{ϵ}_{2}}{{ϵ}_{2} - 1}}$$

with the behavior

$${lim}_{{ϵ}_{2} \to 1} {T}_{{ϵ}_{2}} ( x , y ) = xy and {lim}_{{ϵ}_{2} \to 0} ( x , y ) = min ( x , y ) .$$

Put another way, the rules module 204 can modulate the defined T-norm via ∈₂. In some implementations, the rules module 204 calculates the firing strength rₖ by applying the T-norm to multiple inputs. As such,

$${r}_{k} = {T}_{{ϵ}_{2}} ( {\tilde{x}}_{1 , k}^{{M}_{1 , k}} , {\tilde{x}}_{2 , k}^{{M}_{2 , k}} , … , {\tilde{x}}_{H , k}^{{M}_{H , k}} ) = {g}_{{ϵ}_{2}}^{- 1} ( {\sum }_{i = 1}^{H} {g}_{{ϵ}_{2}} ( {\tilde{x}}_{H , k}^{{M}_{H , k}} ) ) = {( {\sum }_{i = 1}^{H} {\tilde{x}}_{i , k}^{{M}_{i , k} \cdot ( \frac{{ϵ}_{2} - 1}{{ϵ}_{2}} )} - H + 1 )}^{\frac{{ϵ}_{2}}{{ϵ}_{2} - 1}} .$$

In some such implementations, the rules module 204 uses the learned entries in the connection matrix M to vary the contribution and/or weight of each input for a given rule within the T-norm calculation. Further, the lower value in M indicates a lower contribution to the rule firing strength. For example, for

$${\tilde{x}}_{1 , k}^{{M}_{1 , k}}$$

a lower M_{1,k} (e.g., closer to 0) means

$${\tilde{x}}_{1 , k}^{{M}_{1 , k}}$$

is closer to 1 and consequently contributes less to rₖ with the defined T-norm.

The inference module 206 classifies the variables based on the rule firing strength that the rules module 204 calculates. In some implementations, the inference module 206 classifies the variables into C classes. In further implementations, the inference module 206 includes C nodes, one for each class, that are fully connected to nodes of the rules module 204. The inference module 206 calculates the firing strength of each node o_{c} using the rule firing strengths rₖ with an inference matrix W of dimension K×C. An entry W_{j,c} denotes the contribution of the kth rule to the cth class. In some implementations, entries in the inference matrix are all trainable and positive. In further implementations, a higher value in the inference matrix indicates a higher contribution.

In some implementations, the inference module 206 defines a parametrized T-conorm to calculate o_{c}. In particular, the inference module 206 defines the parametrized T-conorm on two inputs as

$${Q}_{{ϵ}_{3}} ( x , y ) = {( {x}^{\frac{1}{{ϵ}_{3}}} + {y}^{\frac{1}{{ϵ}_{3}}} )}^{{ϵ}_{3}} ,$$

where 0<∈₃<1. The T-conorm has asymptotic behavior according to the following:

$${lim}_{{ϵ}_{3} \to 1} {Q}_{{ϵ}_{3}} ( x , y ) = x + y and {lim}_{{ϵ}_{3} \to 0} {Q}_{{ϵ}_{3}} ( x , y ) = max ( x , y ) .$$

Accordingly, the inference module 206 can modulate the defined T-conorm between addition and max by modifying ∈₃. In some such implementations, the inference module 206 then calculates o_{c} according to

$${o}_{c} = {Q}_{{ϵ}_{3}} ( {W}_{1 , c} {r}_{1} , {W}_{2 , c} {r}_{2} , … , {W}_{K , c} {r}_{K} ) = {( {\sum }_{k = 1}^{K} {( {W}_{k , c} {r}_{k} )}^{\frac{1}{{ϵ}_{3}}} )}^{{ϵ}_{3}} .$$

In some further such implementations, after the inference module 206 calculates o₁, o₂, . . . , o_{c}, the inference module 206 applies a softmax activation function to generate probabilities p₁, p₂, . . . , p_{c} of being in each class, which are all in [0,1] with

$${\sum }_{c = 1}^{C} {p}_{c} = 1.$$

Because the softmax activation functions guarantees that

$${\sum }_{c = 1}^{C} {p}_{c} = 1 ,$$

the number of valid nodes in the inference module 206 can be set to C−1 to avoid ambiguity in rule representation. For example, when performing binary classification W_{:,0} can be set to 0 so that the model will only learn subspaces related to the positive class.

Using the encoding module 202, the rules module 204, and the inference module 206, the ML application 201 can both extract and inject fuzzy rules. Put another way, the ML application 201 can extract and inject rules in a way that humans can understand. The entries in the attention matrix A and connection matrix M represent the contribution of individual concepts and individual variables to each rule. The entries in the inference matrix W gives the contribution of individual rules to each class.

In some implementations, the ML application 201 constructs a contribution matrix S using the attention and connection matrices A and M. The contribution matrix S expresses the contribution of individual concepts to each rule in the model. The matrix S is of the same dimension as attention matrix A. Put another way, the matrix S is a partition matrix formed by concatenating submatrices S₁, S₂, . . . , S_{H}. In some implementations, the ML application 201 treats ordinal and continuous variables different from categorical variables. In such implementations, for an ordinal or continuous variable xᵢ, the corresponding submatrix Sᵢ has dimension 3×K. Similarly, for a categorical variable xⱼ, Sⱼ has dimension Lⱼ×K. The ML application 201 calculates entries S_{i,d,k} of Sᵢ and S_{j,d,k} of Sⱼ as S_{i,d,k}=A_{i,d,k}×M_{i,k}, d∈{1,2,3} and S_{j,d,k}=A_{j,a,k}×M_{j,k}, d∈{1, 2, . . . , Lⱼ}, where k∈{1, 2, . . . , K}. The entry S_{i,d,k} is the contribution of the dth concept of xᵢ to the kth rule. S_{:,:,k} encodes the construction of the kth rule, while W_{k,:} captures the relationship between classes and the kth rule. Further, while the ML application 201 is described as calculating S_{i,d,k} using 3 fuzzy concepts and thereby limiting d∈{1,2,3}, it will be recognized that the ML application 201 can use any appropriate number of fuzzy concepts as described in detail above.

As an example of how the ML application 201 generates, modifies, and/or represents humanly understandable rules, the ML application 201 receives a dataset with four continuous input variables x₁, x₂, x₃, x₄ and determines a binary response (negative and positive). A, M, and W are trained and the ML application 201 can calculate S. Further, take entries S_{1,1,1}, S_{2,3,1}, S_{2,2,2}, and S_{3,1,2} of the contribution matrix S as close to 1, with all other entries of S close to 0. In the inference matrix W, W_{1,2} and W_{2,2} are close to 1 while W_{1,1} and W_{2,1} are close to 0. From the given S and W, the ML application 201 can summarize two rules from the trained network as follows: (1) If x₁ is low and x₂ is high, then the sample is positive; (2) If x₂ is medium and x₃ is low, then the sample is positive. Each rule is represented in (S_{:,:,1}, W_{1,:}) and (S_{:,:,2}, W_{2,:}), respectively. The ML application 201 can extract the definitions of low, medium, and high concepts from the parameters in the encoding module 202. The extracted rules mimic human logic, and a user can use the extracted rules to justify the network decisions. In some implementations, the rules are not extracted, but instead the trained model is used as a neural network after being trained, as described in more detail below.

The model training module 208 trains the modules and algorithms described above. In some implementations, the model training module 208 trains the various modules and algorithms by back-propagation with an Adam optimizer. In further implementations, the model training module 208 trains the classification model using a calculated regular cross-entropy loss, loss_{ce}. In still further implementations, the model training module 208 adds an l₁ norm-based regularization term lossₗ_{₁} to the loss function to favor rules with a smaller number of concepts, which are more feasible to use in practice. Further, the model training module 208 calculates the correlation among encoded rules as a loss term loss_{corr} to avoid extracting redundant rules. The loss function is defined as: lossₜₒₜₐₗ=loss_{ce}+λ₁lossₗ_{₁}, +λ₂loss_{corr}; lossₗ_{₁}=∥vec(A)∥₁+∥vec(M)∥₁; and

$${loss}_{corr} = {\sum }_{i = 1}^{H - 1} {\sum }_{j = i + 1}^{H} vec ( {S}_{: , : , i} ) vec ( {S}_{: , : , j} ) ,$$

where λ₁ and λ₂ control the magnitude of the l₁ norm-based regularization term and correlation based regularization term, respectively. vec(·) denotes the vectorization of a matrix.

In some implementations, the ML application 201 constrains ∈₁, ∈₂, ∈₃ to be equal for simplicity. For example, the ML application 201 may initialize each of the three as 0.99 at the beginning of training and gradually reduce each of ∈₁, ∈₂, ∈₃ with the number of training steps. In some implementations, the scheduling of the e values is defined as ∈=max(∈ₘᵢₙ,∈·γ^{training_steps}), where γ00 is the decay rate that can be tuned as a hyperparameter. ∈ₘᵢₙ is another hyperparameter, whose optimal value varies with different applications. In some implementations, the ML application 201 initializes ∈ to ∈=0.99 and reduces e to improve model optimization as discussed in more detail below.

In some implementations, before model training, the model training module 208 randomly initializes training parameters. In further implementations, the model training module 208 may use practical rules from the data processing database 210, the server computing device 102, the rules database 116, and/or the experimental database 118 to improve and/or initialize the network parameters. For example, clinician expertise data associated with stroke management procedure for patients who have been diagnosed with atrial fibrillation. Using such rules to initialize the parameters may improve performance, particularly when the size of the training dataset is small. In the example detailed above, take the generated rules as previously known rules within the rules database 116. In such an example, the model training module 208 could then initialize the matrices A, M, and W as: (1) A: A_{1,1,1}, A_{2,3,1}, A_{2,2,2}, A_{3,1,2} having a higher value and other entries in A_{:,:,1} and A_{:,:,2} having a lower value; (2) M: M_{1,1}, M_{2,1}, M_{2,2}, M_{3,2} having a higher value and other entries in M_{:,1} and M_{:,2} having a lower value; (3) W: W_{1,2}, W_{2,2} having a high value and W_{1,1}, W_{2,1} having a low value; and (4) other entries in A, M, and W being randomly initialized.

In some further implementations, the model training module 208 assesses and/or evaluates the machine learning model after each iteration of training. For example, the model training module 208 may calculate an accuracy, precision, recall, F₁ value (i.e., the harmonic means of precision and recall), and/or the area under the receiver operating characteristic curve. In some such implementations, the model training module 208 calculates the evaluation metrics as follows:

$$accuracy = \frac{true positive + true negative}{all positives + all negatives} ;$$

$$precision = \frac{true positives}{true positives + false positives} ;$$

$$recall = \frac{true positives}{true positives + false negatives} ; and$$

$${F}_{1} = 2 ( \frac{recall * precision}{recall + precision} ) .$$

In further implementations, the model training module 208 calculates generalization gaps as the differences between metrics on validation and test sets. In such implementations, a higher generalization gap indicates greater overfitting.

Depending on the implementation, the model training module 208 outputs the trained model, which a user may use as a neural network to analyze input data. In some implementations, the model training module 208 outputs the trained model in addition to or in place of the trained ruleset. In implementations in which the model training module 208 outputs the trained model in place of the trained ruleset, the trained model may still use the trained ruleset to analyze input data and/or perform other functions as described herein.

In some implementations, the application architecture 200 performs the module functions as outlined above using one or more algorithms and/or a neural network. In further implementations, the application architecture 200 performs the module functions as outlined above to train one or more algorithms and/or a neural network. In some such implementations, to train the algorithms and/or neural network, the model training module 208 uses training data to improve the functionality of the modules and/or the models in question. In some implementations, the model training module 208 trains the algorithms and/or neural network using a supervised machine learning program or algorithm. The neural network may be a convolutional neural network, a deep learning neural network, or a combined learning module or program that learns in two or more features or feature datasets (e.g., determining the coordinates and classification for input data) in a particular area of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the machine learning based algorithms may be included as a library or package executed on a computing platform (e.g., server computing device 102). For example, libraries may include the TENSORFLOW based library, the PYTORCH library, and/or the SCIKIT-LEARN Python library.

Machine learning may involve identifying and recognizing patterns in existing data (such as training a neural network based on labeled classes and training data) in order to facilitate making predictions or identification for subsequent data (such as using the neural network on new input data in order to generate new rulesets, improve old input rulesets, and/or condense rulesets).

Machine learning model(s) implemented on the neural network(s), such as the encoding module 202, rules module 204, and inference module 206 or the models created by the aforementioned modules, described herein for some embodiments, may be created and trained based upon example data (e.g., “training data” and related input data and/or input rules) inputs or data (which may be termed “features” and “labels”) in order to make valid and reliable predictions for new inputs, such as testing level or production level data or inputs. In supervised machine learning, a machine learning program operating as a neural network on a server, computing device, or otherwise processor(s), may be provided with example inputs (e.g., “features”) and their associated, or observed, outputs (e.g., “labels”) in order for the machine learning program or algorithm in the neural network to determine or discover rules, relationships, patterns, or otherwise machine learning “models” that map such inputs (e.g., “features”) to the outputs (e.g., “labels”), for example, by determining and/or assigning weights or other metrics to the model across its various feature categories. Such rules, relationships, or models may then be provided with subsequent inputs for the neural network, executing on the server, computing device, or processor(s), to predict, based on the discovered rules, relationships, or models, an expected output.

In an example implementation, the ML application 201 may utilize a fuzzy machine learning model to refine and/or apply a stroke management ruleset specifically tailored for a population of patients (e.g., patients who have experienced more than one stroke). By training the fuzzy model on historical medical data, including various stroke management procedures/treatments and their outcomes, the application 201 may generate a trained fuzzy ruleset for stroke classification and stroke management recommendations. Further, such trained fuzzy rulesets may provide interpretable stroke management classifications and recommendations that can be generated for real-time or near-real-time clinical data including medical imaging data for the patient, such as MRI data and CT imaging data.

In an example implementation, the application architecture 200 may be set up and implemented as follows. A 10-fold cross-validation may be used to evaluate model performance. For each iteration, the dataset can be split into training, validation, and test sets. A random search algorithm can be applied using the training set and validation set for hyperparameter tuning, including learning rate, batch size, λ₁, λ₂, and ∈ₘᵢₙ.

Compared to popular “black box” machine learning algorithms—such as random forest, SVM, and XGBoost—and other interpretable models—such as logistic regression, decision tree, and Explainable Boosting Machine (EBM)—evaluated using a 10-fold cross-validation, the techniques as described herein provide better accuracy, recall, precision, F1, and area under the ROC curve (AUC) than almost all such models while still providing transparency. Moreover, the techniques described herein have significantly lower generalization error than such black box methods.

In an example implementation, the application architecture 200 receives eight input variables: x₁~N(0,2), x₂~N(5,3), x₃~N(−1,5), x₄~N(1,2), x₅~N(−2,1), x₆~Bernoulli (0,5), x₇~N(0,1), and x₈~N(0,1). Further, the following rules are true for the dataset. Should any of the rules apply to an observation, then the observation is positive and otherwise is negative: Rule A: x₂<3.8 and x₃>−2 and x₆=1; Rule B: x₂>6.3 and x₃>−2 and x₆=1; Rule C: x₁<1 and x₄>2 and x₆=0; Rule D: x₃>0 and x₅>−1 and x₆=0; and Rule E: x₁>1 and x₅>−1.5 and x₆=0. In some further example implementations, random noise sampled from N(0,0.01) is added to the input variables. Further, the application architecture 200 determines that the observations do not rely on x₇ and x₈, so the application architecture 200 deems the variables in question irrelevant.

In the above example, the application architecture 200 receives 400 samples from the dataset, the percentage of positive samples is 34.25%, and the percentages of samples with Rule A, Rule B, Rule C, Rule D, and Rule E are 8.25%, 7.50%, 9.00%, 10.75%, and 2.00%, respectively. The application architecture 200 generates a visual rule summary (e.g., graphical user interface 600a of FIG. 6A) of a summarized ruleset generated using the input dataset. For example, the visual rule summary may indicate that Rule 1 corresponds with Rule C, Rule 2 corresponds with a union of Rule A and Rule B, Rule 3 corresponds with Rule D, and Rule 4 is like Rule E. As such, the application architecture 200 generates most of the rules accurately and generates the final rule similarly due to only 2.00% of samples being consistent with Rule E.

In another example implementation, the application architecture 200 receives nine input variables x₁~N(0,2), x₂~N(5,3), x₃~N(−1,5), x₄~N(1,2), x₅~N(−2,1), x₆~N(−1,4.4), x₇~N(0,1.2), x₈~N(0,1), and x₉~N(0,1), and the sample is positive when

$$\frac{( {x}_{1} + 0.5 {x}_{2} + {x}_{3} )}{1 + {e}^{{x}_{6}} + 2 {x}_{7}} < 1.$$

In the example implementation, the application architecture 200 receives 400 samples from the dataset. A generated ruleset visualization may demonstrate that Rule 1 shows that “high” levels of x₆ and x₇ lead to a positive class, since 1+e^{x6}+2x₇ becomes larger, and thus more likely to cause the expression to be less than 1. Similarly, Rules 4 and 5 illustrate that a low x₃ and a high x₁ or a low/medium x₁ and medium x₃ can similarly lead to a positive class, as (x₁+0.5x₂+x₃)² becomes smaller.

Referring next to FIG. 3A, a diagram 300a illustrates methods for receiving an input dataset and extracting rules therefrom as well as for receiving a trained model and dataset and subsequently developing a summarized ruleset. The method 310 of FIG. 3A may be implemented in application architecture 200 as described with regard to FIG. 2 above. Though the method 310 is described below regarding application architecture 200 and the computing environment 100, it will be recognized that any similarly suitable application and/or environment may be used to implement the method of FIG. 3A.

First, when extracting rules from data and/or training a ruleset using method 310, ML application 201 receives input data 312 from any of and/or any combination of the server computing device 102, the clinician computing device 104 (e.g., input data received via the input/output devices 174), the EHR database 110, the medical imaging device(s) 108, and/or one or more additional computing devices. In some implementations, the input data comprises ordinal variables, continuous variables, categorical variables, crafted rules, or any other similar data as described herein. Depending on the implementation, the ML application 201 may receive the input data 312 directly at an encoding module 314, which may be the encoding module 202 as described regarding FIG. 2 above.

The encoding module 314 then encodes membership values 316 for fuzzy concepts. In some implementations, the encoding module 314 assigns a membership value 316 in the range of [0,1] to each variable and/or component of the input data 312 based on the observed value for a given fuzzy set, indicating the confidence of the variable belonging to a given concept and/or set (i.e., with 0 referring to no confidence and 1 referring to complete confidence). In some implementations, the encoding module 314 uses membership functions to calculate the membership values 316. The encoding module 314 then transmits the membership values 316 to the rule module 318. In some implementations, the rule module 318 is the rules module 204 as described regarding FIG. 2 above.

The rule module 318 then generates a ruleset and/or determines the most relevant concept from each variable for each rule. In some implementations, the rule module 318 further calculates a firing strength for each rule and/or a weight 320 for each generated rule. Put another way, the rule module 318 determines which rules of the ruleset have the greatest effect on the outcome. In some implementations, the rule module 318 determines the firing strength and/or weight 320 for each rule by a weighting system, such that each rule has potential to affect the outcome in accordance with the weight of the respective rule. In further implementations, the rule module 318 determines the firing strength and/or weight 320 for each rule by a priority system, such that the applicable rule with the greatest priority controls.

In some implementations, the rule module 318 generates a piecewise categorizing function based on the generated ruleset and/or the firing strength/weight for each rule. In particular, the piecewise categorizing function covers the generated ruleset and details the relationships between various input variables and the output according to the generated ruleset. After generating the piecewise categorizing function representative of the ruleset, the rule module 318 may approximate a continuous representation of the piecewise categorizing function using tropical geometry, such as a gradient descent function. In other implementations, the inference module 322 approximates the continuous representation rather than the rule module 318. The rule module 318 then transmits the ruleset, the piecewise categorizing function, the continuous approximation, the firing strength, and/or the weight 320 to the inference module 322. In some implementations, the inference module 322 is the inference module 206 as described in FIG. 2 above.

The inference module 322 then classifies the variables based on the rule firing strength and/or weights 320 that the rule module 318 calculates. In some implementations, the inference module 322 further determines the firing strength of each class on the output using the rule firing strength and/or weights 320. In some implementations, the output 324 includes the classified variables and/or the class firing strength of the classes. In further implementations, the output 324 includes a trained model as described herein. In still further implementations, the output 324 is a basic model that a module, such as rule training module 208, trains.

The ML application 201 may also perform a rule summary method 330. As part of the rule summary method 330, the ML application 201 uses trained models 332, such as those described above regarding output 324 and FIG. 2, and/or a dataset 334. In some implementations, the ML application 201 already has access to the trained models 332 and/or dataset 334, such as from the inference module 322 or from an internal database 210. In other implementations, the ML application 201 receives the trained models 332 and/or dataset 334 via the server computing device 102, the clinician computing device 104, the rules database 116, and/or the experimental database 118. Though FIG. 3A illustrates trained models, it will be understood that the ML application 201 may similarly perform the rule summary method 330 before or as part of training the models at the rule training module 208.

After receiving and/or retrieving the trained models 332 and/or dataset 334, the ML application 201 performs a distance matrix calculation 336. In some implementations, the ML application 201 performs the distance matrix calculation 336 by calculating weights for individual variables to individual rules. The ML application 201 then averages the calculated weights over all data samples in the dataset to construct a matrix A with a size in accordance with the number of variables and the number of rules. For example, the matrix A may have size n×m, where n is the number of variables and m is the number of rules. The ML application 201 then calculates the distance matrix based on the matrix A. In some implementations, the distance matrix is D=1−A^{T}A, where an entry d_{i,j} indicates the distance between rule i to rule j. In some implementations, the ML application 201 performs distance matrix calculation using the piecewise categorizing function representing the generated ruleset to generate a continuous representation of the piecewise categorizing function.

After performing the distance matrix calculation 336, the ML application 201 clusters 338 the rules into groups. In some implementations, the ML application 201 clusters 338 the rules using a hierarchical clustering technique. In further implementations, the hierarchical clustering technique uses an agglomerative or bottom-up approach, where each rule starts with a cluster and the ML application 201 successively merges similar clusters, minimizing the distance between pairs of clusters. In other implementations, the hierarchical clustering technique uses a divisive or top-down approach, where the rules start as a single cluster and the ML application 201 successively divides the cluster into similar, smaller clusters.

After clustering 338 the rules, the ML application 201 performs a representative rule selection 340. In some implementations, the ML application 201 selects representative rules from each cluster. In further implementations, after selecting the representative rules, the ML application 201 discards any remaining rules. In other implementations, the ML application 201 stores the remaining rules and/or a reference to the remaining rules in the database 210. Depending on the implementation, the ML application 201 may select the representative rules from each cluster based on the firing strength and/or weight 320 of each rule. For example, the ML application 201 may select the rule with the greatest weight 320 or contribution to the classification. In some implementations, the data processing system calculates the contribution and/or weight 320 of each rule as the average of the weights for a rule over data samples in the dataset. In further implementations, the ML application 201 performs or confirms representative rule selection 340 by iteratively determining a local minimum and/or maximum of the ruleset using a gradient descent algorithm and modifying the overall ruleset based on such. For example, depending on the implementation, the ML application 201 can remove any rules resulting in local minima or maxima and/or keeping only rules that result in local minima or maxima to construct a continuous approximation of the piecewise categorizing function representative of the ruleset. The ML application 201 then outputs the set of summarized rules 342 as summarized by the rule summary method 330.

Referring next to FIG. 3B, a diagram illustrates further systems and methods for receiving an input dataset and extracting rules therefrom. The system 300b may implement methods as described regarding FIG. 7 below. Though the system 300b is described below with reference to the application architecture 200 and the computing environment 100, it will be recognized that any similarly suitable system may be used to implement FIG. 3B.

The diagram illustrates a system 300b including multiple modules and layers for analysis. In particular, the system 300b includes an input layer 350, encoding module 360, rule module 370, and inference module 380. In some implementations, the inference module 380 also serves as an output layer and may be referred to as such herein. In further implementations, the system 300b is part of application architecture 200 and may be the ML application 201. Similarly, each of the encoding module 360, rule module 370, and inference module 380 may include or be each of the encoding module 202, rules module 204, and inference module 206, respectively.

At the input layer 350, the system 300b receives input datasets. In some implementations, the input datasets include ordinal variables, continuous variables, categorical variables, rules, trained rules, and/or any combination thereof. In further implementations, the input layer identifies and separates ordinal and continuous variables 352 xᵢ from categorical variables 354 xⱼ. Though FIG. 3B illustrates two variables, it will be understood that this is for ease of illustration and understanding, and the system may receive any number of input variables divided in any proportion between ordinal and continuous variables 352, categorical variables 354, and/or other forms of input data.

The encoding module 360 receives the input data from the input layer 350 and encodes the input data into one or more fuzzy concepts. In some implementations, the encoding module 360 encodes the input data differently based on the variable type. For example, the encoding module 360 may encode ordinal and/or continuous variables 352 based on a predetermined number of fuzzy concepts corresponding to FIG. 2. Similarly, the encoding module 360 may encode categorical variables 354 regarding the number of layers Lⱼ each variable has, as described regarding FIG. 2.

In the exemplary embodiment of FIG. 3B, the encoding module 360 encodes ordinal and/or continuous variables 352 into three fuzzy concepts, a low concept 362, a medium concept 364, and a high concept 366. In some implementations, the fuzzy concepts are the low concept

$$362 l ( {x}_{i} ) = {f}_{{ϵ}_{1}} ( \frac{{a}_{i , 2} - x}{{a}_{i , 2} - {a}_{i , 1}} ) - {f}_{{ϵ}_{1}} ( \frac{{a}_{i , 1} - x}{{a}_{i , 2} - {a}_{i , 1}} ) ,$$

the medium concept

$$364 m ( {x}_{i} ) = {f}_{{ϵ}_{1}} ( \frac{x - {a}_{i , 1}}{{a}_{i , 2} - {a}_{i , 1}} ) - {f}_{{ϵ}_{1}} ( \frac{x - {a}_{i , 2}}{{a}_{i , 2} - {a}_{i , 1}} ) - {f}_{{ϵ}_{1}} ( \frac{{a}_{i , 3} - x}{{a}_{i , 4} - {a}_{i , 3}} ) + {f}_{{ϵ}_{1}} ( \frac{{a}_{i , 4} - x}{{a}_{i , 4} - {a}_{i , 3}} ) - 1 ,$$

and the high concept

$$366 h ( {x}_{i} ) = {f}_{{ϵ}_{1}} ( \frac{x - {a}_{i , 3}}{{a}_{i , 4} - {a}_{i , 3}} ) - {f}_{{ϵ}_{1}} ( \frac{x - {a}_{i , 4}}{{a}_{i , 4} - {a}_{i , 3}} ) , where {f}_{{ϵ}_{1}} ( x ) = {ϵ}_{1} log ( 1 + exp ( \frac{x}{{ϵ}_{1}} ) )$$

tunable hyperparameters a_{i,1}<a_{i,2}<a_{i,3}<a_{i,4}, and 0<∈₁<1 as defined above. In further implementations, the encoding module 360 encodes the categorical variables 354 into encoded concepts 368A-368L, defined as l₁(xⱼ), . . . , l_{L}_{ⱼ}(xⱼ), where only one of l₁(xⱼ), . . . , l_{L}_{ⱼ}(xⱼ) has a value of 1, while all others have a value of 0.

The rule module 370 determines a firing strength of each variable on each rule as well as the firing strength of each rule. In particular, the rule module determines a vector \tilde{x}ᵢ 372 comprising entries 372A-372K \tilde{x}_{i,1} to \tilde{x}_{i,K} for variable 352 xᵢ, as well as the corresponding vector 376 \tilde{x}ⱼ and entries 376A-376K \tilde{x}_{j,1} to \tilde{x}_{j,K} for variable 354 xⱼ. In some implementations, the rule module 370 determines the vectors and entries based on a determined attention matrix A and the corresponding entries for each concept and each entry of the vectors \tilde{x}ᵢ and \tilde{x}ⱼ. For example, the low concept 362 may represent the contribution of the variable x¿ 352 on a first rule entry vector by the attention matrix entry A_{i,l,1} and on a Kth rule entry by the attention matrix entry A_{i,l,K}. Similarly, the encoded concept 368A may represent the contribution of the ordinal variable xⱼ 354 on a first rule entry by the attention matrix entry A_{j,1,1} and on a Kth rule entry by the attention matrix entry A_{j,1,K}. In some implementations, the rule module 370 uses the attention matrix entries to generate a piecewise categorizing function representative of the ruleset. For example, the rule module 370 may use the entries to determine what fuzzy concepts a variable xᵢ contributes positively to the output and generate the piecewise categorizing function based on such.

The rule module 370 further determines the firing strength r₁, . . . , r_{K} 374A-374K based on a connection matrix M as described in more detail regarding FIG. 2 above. An entry M_{i,k} in the connection matrix denotes the contribution of the variable to the kth rule, constructed from the rule entry vectors \tilde{x}_{1,k}, . . . , \tilde{x}_{H,k}. In particular, the firing strength of a rule K, r_{K} 374K, depends on the corresponding entry for each connection matrix, constructed for each variable. Put another way, M_{i,K}, M_{j,K}, and every other such matrix determines the firing strength r_{K} 374K as described regarding FIG. 2 above.

The inference module 380 determines a firing strength for each class into which the system 300b classifies the variables. In particular, the inference module determines class firing strengths o₁, . . . , o_{c} 382A-382C. In some implementations, the inference module 380 determines the class firing strengths 382A-382C based on an inference matrix W. In some such implementations, each rule firing strength 374A-374K affects each class firing strength 382A-382C. For example, the rule firing strength r₁ 374A has a contribution on each class firing strength 382A-382C in accordance with the matrix entries W_{1,1}, . . . , W_{1,C}.

Referring next to FIG. 4, a block-flow diagram illustrates a method 400 for generating a predicted outcome following a cerebrovascular accident. The method 400 of FIG. 4 may be implemented by the processors 130, the processors 180, and/or other suitable processors, etc., executing instructions stored on the memories 140, the memories 190, and/or another suitable non-transitory computer readable medium, etc., described above with respect to FIG. 1. Though the method below is described regarding the computing environment 100, it will be recognized that any similarly suitable environment(s) and/or system(s) may be used to implement method 400.

As described above, the memories 140 of the server computing device 102 may include one or more modules such as an ML training module 142, an ML operation module 144, an image processing module 146, a classification module 148. Generally, the method 400 may include obtaining or receiving medical imaging data (block 402) at the image processing module 146, as described above with respect to FIG. 1 (e.g., the medical imaging data may be provided to or obtained from a component of the computing environment 100, such as the medical imaging device 108 or the EHR database 110). For example, the medical imaging data 402 may correspond to a cerebrovascular accident associated with a patient. The image processing module 146 may apply preprocessing operations to the medical imaging data 402, such as mapping the medical imaging data to a standardized coordinate system and normalizing the medical imaging data using a brain mask (e.g., as described above with respect to FIG. 1). The method 400 may further include providing the preprocessed imaging data 402 to the ML operation module 144.

The ML operation module 144 may provide the medical imaging data 402 to one or more autoencoders (block 404). For example, an autoencoder 404 may be a machine learning model, such as an autoencoder, trained (e.g., via ML training module 142 as described above with respect to FIG. 1) on historical medical images of a particular modality, anatomical region, demographic of patient, etc., to generate embedding vectors and reduce the dimensionality of input feature vectors. In some embodiments, an example autoencoder may be a ML model trained (e.g., via ML training module 142 as described above with respect to FIG. 1) on historical clinical/medical information such as electronic health record (EHR) data, procedural data, clinical guidelines, clinician notes, medical interventions and associated outcomes, etc., and historical medical images associated with a patient population including the patient, to generate contextual information (e.g., relevant clinical information) associated with input medical imaging data.

Furthermore, the method 400 may include generating one or more embedding vectors (block 410) using the one or more autoencoders 404. For example, the medical imaging data 402 may include multi-modal imaging data (e.g., MRI data and CTA imaging data) and the ML operation module 144 may provide the medical imaging data 402, or a portion of the imaging data, to a first feature extraction model trained for a first modality (e.g., MRI data), a second feature extraction model trained for a second modality (e.g., CTA imaging data), etc.

Additionally, the method 400 may include generating contextual information (block 412) using an autoencoder. For example, an additional autoencoder (not depicted) may be trained (e.g., via ML training module 142) on historical medical images and EHR data for the corresponding patient population and an additional autoencoder (e.g., one of the autoencoders 404) may be trained (e.g., via ML training module 142) on historical medical images and other clinical data, such as procedural data and clinical guidelines.

The method 400 may include providing the embedding vector(s) 410 and the contextual information 412 to one or more ML classifier models (block 420). As described above, the ML training module 142 may train one or more ML classifier models to generate stroke outcome classifications for medical imaging data. Further, the ML operation module 144 may generate one or more classifications (block 430) for the medical imaging data 402 by inputting the one or more embedding vectors 410 and the contextual information 412 to the one or more ML classifier models 420. For example, a first ML classifier of the one or more ML classifiers 420 may be trained to generate a length of stay (LOS) classification (block 432) for input embedding vectors and a second ML classifier may be trained to generate a modified Rankin scale (mRS) classification for input embedding vectors. As described above with respect to FIG. 1, an example ML classifier may be trained to generate a LOS classification (e.g., predicted timelines for: recovery of motor functions, recovery of speech functions, predicted time to ambulation), a mRS classification (e.g., a classification measuring the degree of disability or dependence in daily activity of a patient), a discharge classification (e.g., discharge to home, discharge to inpatient rehabilitation, discharge to nursing facility, etc.), an intervention classification (e.g., need for a tracheostomy, need for a PEG tube, etc.), and/or a combined or aggregated classification (e.g., incorporating features and/or concepts from the other classifications). In would be understood that the method 400 may include generating any number of such classifications for the medical imaging data 402.

The classification module 148 may obtain or receive the classifications 430 from the ML classifier 420 including, for example, a LOS classification 432 and an mRS classification 434. The method 400 may include providing the classification 430, 432, and 434 to the clinician computing device 104 (e.g., via the network 106). For example, the classification module 148 may generate one or more graphical representation and/or other visualizations of the generated classifications, as described below with respect to FIGS. 3-7. In some embodiments, the method 400 includes providing the medical imaging data 402 and the contextual information 412 to the clinician computing device 104 (e.g., to be reviewed by a clinician) with, for example, the classifications 430, 432, 434.

Referring next to FIG. 5, a block-flow diagram illustrates an architecture 500 for an ensemble machine learning model. The model architecture 500 of FIG. 5 may be implemented by the processors 130, the processors 180, and/or other suitable processors, etc., executing instructions stored on the memories 140, the memories 190, and/or another suitable non-transitory computer readable medium, etc., described above with respect to FIG. 1. Though the method below is described regarding the computing environment 100, it will be recognized that any similarly suitable environment(s) and/or system(s) may be used to implement the model architecture 500.

The architecture 500 may process medical imaging data through a multi-step encoding, feature extraction, augmentation, and classification pipeline. Initially, medical imaging data 402, or x₁, may be provided to a first autoencoder 502. The autoencoder 502 employs an encoding-decoding structure (e.g., encoding the initial input data x₁ into a lower-dimensional latent representation z₁). The latent representation z₁ may encapsulate underlying structural and contextual features of the medical imaging data 402, thereby facilitating more robust downstream tasks such as clinical feature extraction and classification.

The latent features z₁ generated by the first autoencoder 502 may be provided as input to a second autoencoder 504. Additionally, or alternatively, the initial input data x₁ may be provided as input to the second autoencoder 504. Like autoencoder 502, the second autoencoder 504 may employ an encoding-decoding mechanism to further refine or extract latent features z₂ (e.g., deeper, more context-rich latent features, relative to latent features z₁). In some embodiments, the latent features z₁ may be combined with additional feature vectors, or embeddings, of varying medical imaging modalities (e.g., generated by one or more additional trained autoencoders), when provided as an input to autoencoder 504. Furthermore, the selection of x₁ and/or z₁ as the input to autoencoder 504 supports multiple strategies for representation learning, allowing optimized tuning depending on the intended clinical scenario or classification task.

The refined latent features z₂ generated by the second autoencoder 504 may be provided as input to a fuzzy neural network model 508 (e.g., an FNN model trained on partially available privileged information, as described above with respect to FIGS. 1-4). Additionally, clinical data x₂, or clinical features 506 (e.g., contextual information 412 of FIG. 4), may be provided to the FNN model 508. For example, the clinical data x₂ may correspond to clinical target labels (e.g., clinical outcomes), such as clinically derived ground truth information. When the latent features z₁ are combined with the additional features of varying imaging modalities, the refined latent features z₂ generated by the second autoencoder 504 may encode features from each modality of the features provided an input to autoencoder 504.

For example, if a patient has MRI imaging data (x1_MRI) and CT imaging data (x1_CT), two separate autoencoders (502_MRI and 502_CT) may be trained. Further, the corresponding embeddings, z1_MRI and z1_CT, of x1_MRI and x1_CT respectively, from the trained autoencoders may be provide as in input to another autoencoder (e.g., autoencoder 504) which compressing multiples embeddings into a single feature vector (e.g., refined latent features z₂). Additionally, the corresponding refined latent features z₂ may be combined with clinical data x₂ and provided as an input to the fuzzy neural network to generate a classification.

Generally, the autoencoder 502 and/or the autoencoder 504 may include convolutional layers and ReLU layers arranged in structured channel depths (e.g., 32, 64, and 128) and/or one or more residual blocks comprising additional convolutional layers, batch normalization layers, and ReLU layers that employ residual connections to enhance feature representation and layer stability. In comparison to conventional approaches, the autoencoders 502, 504 provide improved computational efficiency and generate embedding vectors with richer and more clinically relevant latent features. Further the autoencoders 502 and 504 may be specifically trained on historical imaging data, and in some cases associated clinical context, to generate embedding representations that more effectively capture clinically meaningful feature patterns contained in high-dimensional medical imaging inputs. Additionally, the autoencoder-based approach described herein provides significant operational advantages over general CNN classifier architectures such as residual networks. For instance, due to their modular nature, an example autoencoder requires only a single initial training; in contrast, conventional feature extraction models typically necessitate retraining from scratch for each prognostic outcome of interest, resulting in substantially increased computational overhead. Further, the modular and flexible structure of autoencoders 502, 504 lends itself to implementations leveraging advanced frameworks, such as learning using partially available privileged information (LUPAPI), enabling more robust handling of incomplete patient data.

Subsequently, the outputs(s) from autoencoders 502, 504 (i.e., extracted imaging features/embedding vectors) are provided to a FNN model 508. As discussed below, the FNN model 508 integrates clinically relevant inputs, such as imaging-derived features (IDF) and electronic health record (EHR) data (e.g., obtained from EHR database 110) to generate classifications for medical imaging data. After integrating these multimodal inputs-consisting of the imaging-derived embedding vectors from autoencoders 502, 504 and supplementary data such as EHR data and IDF data (e.g., training data provided to the model, additional context provided to the ML classifier, etc.)—the model may generate a classification 510, for example, predicting stroke outcomes, length of stay (LOS), modified Rankin scale (mRS) scores, or other clinical classifications as described with respect to FIGS. 1-4.

Generally, the FNN model 508 of FIG. 5 may function as a specialized machine learning component configured to integrate and analyze both latent feature representations (e.g., z₂ generated by autoencoder 504) and additional clinical feature inputs or clinical data (e.g., x₂). Specifically, the FNN model 508 may synthesize latent image-derived features z₂ (e.g., embedding vectors 410 of FIG. 4) along with auxiliary clinical features x₂ (such as clinician notes, EHR data, demographic patient information, alternate imaging modalities, patient history, procedural information, or combinations thereof) to generate a classification 510 for the medical imaging data 402. By analyzing both latent image features and clinically relevant data points, the FNN model 508 may leverage multimodal data to generate classification predictions closely aligned with clinical outcomes. The classification 510 may include clinically relevant outcomes such as cerebrovascular accident prognosis, length of stay (LOS) classifications, modified Rankin scale (mRS) classification, intervention recommendations, discharge planning decisions, or similar diagnostic or prognostic predictions.

In some embodiments, the FNN model 508 leverages a strategy referred to as learning with partially available privileged information (LUPAPI). Generally, with LUPAPI, the FNN model 508 may employ privileged clinical information which may be fully accessible during model training (e.g., historical patient EHR data, clinician notes, chronic medical conditions, or additional clinical contextual variables), but only partially or not available at all during inference (e.g., clinical information for a particular patient is missing), when the model processes new patient data. In this manner, the FNN model 508 utilizes fully available privileged knowledge at training time, enabling the encoder and classifier components to learn highly robust and clinically relevant latent and clinical feature representations. As a result, even if some privileged clinical details are incomplete or entirely absent in operational scenarios, such as during clinical decision-making for new stroke cases, the FNN model 508 effectively generalizes to provide reliable predictions based on the available latent imaging-derived features (z₂) and clinical features (x₂) alone.

Furthermore, by employing LUPAPI within the FNN model 508, classification outcomes may be clinically informed by rich historical clinical data sets and advanced medical knowledge accumulated over large patient populations. For example, historical EHR data representing privileged clinical information may be integrated together with latent embedding vectors from historical medical images during the network training phase. This enables advanced artificial neural networks within the FNN model 508 structure to accurately learn intricate mappings between multimodal historical patient data and clinical outcomes. Consequently, even when certain clinical variables or privileged information are only partially available for new patients, predictions remain highly accurate and clinically meaningful, ultimately supporting efficient and reliable clinical interventions, timely patient resource allocation, as well as improved healthcare outcomes.

Generally, an FNN model 508 may generate classification 510 based on latent features z₂ from autoencoder 504 and clinical features x₂. As discussed above, clinical features (e.g., contextual information 412) processed by the FNN model 508 may represent underlying information such as relevant imaging-derived features (e.g., from historical imaging data), patient clinical history, electronic health record (EHR) data, associated medical modalities, or other clinically significant inputs that support robust predictive analysis. Further, by analyzing and integrating latent embedding vectors z₂ and clinical data x₂, the FNN model forms robust clinical representations to generate classifications 510. In summary, the example architecture 500 supports comprehensive medical imaging analysis, enhancing clinical interpretability, predictive accuracy, and diagnostic or prognostic decision-making processes.

Additionally, the ensemble machine learning architecture 500 can leverage parallel deep-feature extraction (via autoencoders 502, 504) and clinical-context generation (via FNN model 508) to analyze complex medical imaging data and clinical context in a coordinated fashion. By integrating multimodal embedding representations (both image-derived and contextually-informed) along with relevant external data sources (EHR data, imaging-derived features, historical patient data), the FNN model 508 may accurately generate clinically actionable predictions for stroke-related patient outcomes (e.g., LOS, mRS, intervention requirement, or discharge decisions).

Referring next to FIG. 6A, example graphical user interface 600a illustrates an example visualization of a ruleset and concepts, as well as contributions to positive classes. More particularly, the example graphical user interfaces (GUI) 600a illustrates a visualization of rules for stroke management for an example patient. In the example GUI 600a, each column represents a stroke management rule, and each row represents one “fuzzy” concept of a clinical feature with the exact values of each concept derived empirically during model training (e.g., as described with respect to FIGS. 1-5). Specifically, the example GUI 600a depicts four columns respectively corresponding to example rule 18 (R18), example rule 17 (R17), example rule 19 (R19), and example rule 20 (R20). The example GUI 600a includes six rows respectively corresponding to fuzzy concepts including legion volume, pre-stroke modified Rankin scale (mRS), initial National Institute of Health stroke scale, age, and blood pressure. The number beneath each rule or column measures the importance of that rule to the model (e.g., R1 has a score of 0.65, R2 has a score of 0.20, R3 has a score of 0.12, R4 has a score of 0.03). For example, stroke management R1 is: “If legion volume is HIGH, then X . . . ”, where X is some type of intervention or outcome prediction. As another example, stroke management R2 is: “If pre-stroke mRS is HIGH or MEDIUM (e.g., not LOW) and age is HIGH or MEDIUM (e.g., not LOW), then Y . . . ”, where Y is some type of intervention or outcome prediction. Additionally, some rules may be more important than other rules and certain fuzzy concepts may be more important than other fuzzy concepts. For example, the GUI 600a depicts that R1 is particularly important to the model with a score of 0.65. Expanding on this example, particular fuzzy concepts within a rule may be more important than others, such as in the case of R2, where pre-stroke mRS may be more important than age, for example.

Referring next to FIG. 6B, a graphical depiction illustrates a classification visualization 600b for a predicted outcome following a cerebrovascular accident. More particularly, the visualization 600b includes a graph showing predicted recovery trajectories after stroke (with best/worst outcomes), with the x-axis representing time after stroke, beginning at an initial time point (e.g., at admission) and progressing through subsequent intervals (e.g., a week, a month, three months, a year, etc.), and the y-axis representing a recovery score (e.g., ranging from 0 to 100) indicative of the level of patient recovery achieved over the indicated time span. For example, a length of stay (LOS) classification, generated as described above with respect to FIG. 1-4, may include recovery scores over various time spans. The visualization 600b may include predictive trajectories for both speech function recovery and motor function recovery, and corresponding trajectory ranges (e.g., best-worst outcome ranges). Lastly, the visualization 600b may include an ambulation milestone, the horizontal line at approximately a recovery score of 50, signifying a critical clinical threshold generally associated with independent ambulation or significant mobility improvement.

Referring next to FIG. 6C, a graphical depiction illustrates a classification visualization 600c for a predicted outcome following a cerebrovascular accident. More particularly, the visualization 600c includes a graph illustrating scenario-based simulations of predicted stroke recovery trajectories for various rehabilitation strategies. The x-axis represents time after stroke and the y-axis represents a recovery score (e.g., ranging from 0 to 100) indicative of the level of patient recovery achieved with the particular rehabilitation strategy. The visualization 600c may include predicted trajectories corresponding to: (1) baseline recovery under standard care, (2) recovery under early intensive rehabilitation, and (3) recovery under delayed or limited rehabilitation.

Referring next to FIG. 6D, a graphical depiction illustrates a classification visualization 600d for a predicted outcome following a cerebrovascular accident. More particularly, the visualization 600d includes a table visualization predicted outcomes and/or classification and recommendations for a patient (e.g., generated based on medical imaging data for the patient as described above with respect to FIG. 1-6). For example, the visualization 600d may include columns for patient factors or clinical categories, generated predictions, and/or recommended interventions. Each row of the visualization 600d may correspond to a specific clinical patient factor. For example, stroke outcome classifications and corresponding treatment recommendations, generated as described above with respect to FIG. 1-5, may be presented in the AI prediction and recommended intervention columns, respectively.

In some embodiments the example graphical user interfaces (e.g., the example GUI 600a of FIG. 6A) and visualizations (e.g., example visualizations 600b-600d of FIGS. 6B-6D, respectively) described herein for presenting classifications and/or rules/rulesets to a user (e.g., a clinician) may include additional indicia, such as indicia indicative of the relative importance of particular fuzzy concepts (e.g., a gradient shading and key) and/or indicia indicative of the relative importance of particular fuzzy rules. Additionally or alternatively, the example graphical user interfaces and visualizations may include further additional indicia, such as indicia indicative of: measures of uncertainty associated with each of the rules and/or model predictions, instances of changing importance of the rules and/or concepts as dictated by changes in the training data and/or context-specific modifications by end users, linkages between the rules and membership functions (e.g., to easily visualize the membership functions of the concepts comprising each rule), and/or situations when data may be missing such that a rule is not applicable. Furthermore, the example graphical user interfaces and visualizations, 600a-600d, of rulesets/concepts and classifications discussed herein with respect to FIGS. 6A-6D, may be presented and/or displayed by a user interface such as the user interface 170 of the clinician computing device 104, in response to the processors 130, the processors 180, and/or other suitable processors, etc., executing instructions stored on the memories 140, the memories 190, and/or another suitable non-transitory computer readable medium, etc., described above with respect to FIG. 1. Though the visualizations and graphical user interfaces above are described regarding the computing environment 100, it will be recognized that any similarly suitable environment(s) and/or system(s) may be used to present the GUI 600a and the visualizations 600b-600c.

Referring next to FIG. 7, a flowchart illustrates a method 700 for assessing medical imaging data for a patient using a trained fuzzy ruleset. The method 700 of FIG. 7 may be implemented by the processors 130, the processors 180, and/or other suitable processors, etc., executing instructions stored on the memories 140, the memories 190, and/or another suitable non-transitory computer readable medium, etc., described above with respect to FIG. 1. Though the method below is described regarding the computing environment 100, it will be recognized that any similarly suitable environment(s) and/or system(s) may be used to implement method 700.

At block 702, a computing device (e.g., server computing device 102) may generate a cerebrovascular accident ruleset based on fuzzy concepts. For example, the fuzzy concepts, and/or the cerebrovascular accident ruleset, may represent clinician expertise data associated with cerebrovascular accident procedures for a patient population. In some embodiments, the method 700 may further include encoding the clinician expertise data into fuzzy concepts, wherein the fuzzy concepts represent approximate logical relationships between variables.

At block 704, the computing device may initialize a fuzzy machine learning model based on the cerebrovascular accident ruleset. For example, the server computing device 102 may initialize a fuzzy neural network (FNN) model, for training, as described above with respect to FIG. 1-5.

At block 706, the computing device may train the fuzzy machine learning model on historical electronic health record (EHR) data for the patient population to generate a trained fuzzy ruleset. In some embodiments, training the fuzzy machine learning model on historical EHR data for the patient population to generate a trained fuzzy ruleset includes training, using the fuzzy machine learning model, the cerebrovascular accident ruleset based on the historical EHR data. For example, training the cerebrovascular accident ruleset (e.g., as described with respect to FIGS. 2-3B) based on the historical EHR data may include approximating a continuous representation of a piecewise categorizing function using tropical geometry, and/or generating the trained fuzzy ruleset based on at least the continuous representation of the piecewise categorizing function.

At block 708, the computing device may generate, via one or more trained autoencoders, at least one embedding vector for medical imaging data of a patient. In some embodiments, the medical imaging data is magnetic resonance imaging (MRI) data, computed tomography (CT) imaging data, computed tomography angiography (CTA) imaging data, and/or another type of medical imaging data suitable for assessing cerebrovascular accidents. In some embodiments, the method 700 may further include training at least one autoencoder (e.g., as described with respect to FIGS. 4-5) of the one or more autoencoders on a historical medical imaging dataset to reduce dimensionality of input feature vectors. For example, imaging data included in the historical medical imaging dataset may be mapped to a standardized coordinate system and normalized by applying a brain mask.

At block 710, the computing device may generate contextual information for the medical imaging data based on the historical EHR data for the patient population. In some embodiments, the historical EHR data includes partially available privileged information for the patient population. Further, the method 700 may further include training an artificial neural network model (e.g., as described with respect to FIGS. 4-5) to learn a function that maps a standard feature space of historical medical image embeddings and a privileged feature space of the historical EHR data to an outcome.

At block 712, the computing device may apply the trained fuzzy ruleset to the at least one embedding vector and the contextual information to generate one or more of: (i) an intervention recommendation for the patient, and/or (ii) a stroke outcome classification for the medical imaging data. For example, the trained fuzzy ruleset may output a stroke classification, for medical imaging data of a patient, which is informed by the generated contextual information (e.g., contextual information generated by a trained autoencoder, as described with respect to FIGS. 4-5). Further, an example rule in the trained fuzzy ruleset may weigh image features derived (e.g., via one or more trained autoencoders) from medical imaging data against clinical features derived (e.g., via one or more trained autoencoders) from clinical data. In some embodiments, the stroke outcome classification is a length of stay (LOS) classification, a modified Rankin scale (mRS) classification, a recovery timeline classification, or an intervention classification. In some embodiments, the method 700 further includes presenting, on a display of a user computing device (e.g., clinician computing device 104), the intervention recommendation or the stroke outcome classification. Additionally, or alternatively, the method 700 may further include presenting, on a display of a user computing device, one or more first indications of corresponding rules included in the trained fuzzy ruleset.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but may also be configured in ways that are not listed.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

Moreover, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality and improve the functioning of conventional computers.

What is claimed is:

1. A computer-implemented method for assessing medical imaging data for a patient using a trained fuzzy ruleset, the method comprising:
generating, via one or more processors, a cerebrovascular accident ruleset based on fuzzy concepts, wherein the cerebrovascular accident ruleset represents clinician expertise data associated with cerebrovascular accident procedures for a patient population;
initializing, via the one or more processors, a fuzzy machine learning model based on the cerebrovascular accident ruleset;
training, via the one or more processors, the fuzzy machine learning model on historical electronic health record (EHR) data for the patient population to generate a trained fuzzy ruleset;
generating, via one or more trained autoencoders, at least one embedding vector for the medical imaging data;
generating, via the one or more processors, contextual information for the medical imaging data based on the historical EHR data for the patient population; and
applying, via the one or more processors, the trained fuzzy ruleset to the at least one embedding vector and the contextual information to generate one or more of: (i) an intervention recommendation for the patient, and (ii) a stroke outcome classification for the medical imaging data.

2. The computer-implemented method of claim 1, wherein training the fuzzy machine learning model on historical EHR data for the patient population to generate a trained fuzzy ruleset includes:
training, via the one or more processors and using the fuzzy machine learning model, the cerebrovascular accident ruleset based on the historical EHR data.

3. The computer-implemented method of claim 2, wherein training the cerebrovascular accident ruleset based on the historical EHR data includes:
approximating, using tropical geometry, a continuous representation of a piecewise categorizing function, and
generating, based on at least the continuous representation of the piecewise categorizing function, the trained fuzzy ruleset.

4. The computer-implemented method of claim 1, further including:
encoding, via the one or more processors, the clinician expertise data into fuzzy concepts, wherein the fuzzy concepts represent approximate logical relationships between variables.

5. The computer-implemented method of claim 1, wherein the historical EHR data includes partially available privileged information for the patient population, and wherein the method further includes:
training, via one or more processors of a ML classifier model, an artificial neural network model to learn a function that maps a standard feature space of historical medical image embeddings and a privileged feature space of the historical EHR data to an outcome.

6. The computer-implemented method of claim 1, further including:
training, via the one or more processors, at least one autoencoder of the one or more autoencoders on a historical medical imaging dataset to reduce dimensionality of input feature vectors,
wherein imaging data included in the historical medical imaging dataset is mapped to a standardized coordinate system and normalized by applying a brain mask.

7. The computer-implemented method of claim 1, wherein the stroke outcome classification is a length of stay (LOS) classification, a modified Rankin scale (mRS) classification, a recovery timeline classification, or an intervention classification.

8. The computer-implemented method of claim 1, wherein the medical imaging data is magnetic resonance imaging (MRI) data, computed tomography (CT) imaging data, or computed tomography angiography (CTA) imaging data.

9. The computer-implemented method of claim 1, further including:
presenting, on a display of a user computing device, the intervention recommendation, or the stroke outcome classification.

10. The computer-implemented method of claim 9, further including:
presenting, on a display of a user computing device, one or more first indications of corresponding rules included in the trained fuzzy ruleset.

11. A computing system comprising:
one or more processors; and
one or more memories including computer-executable instructions that, when executed by the one or more processors, cause the computing system to:
generate a cerebrovascular accident ruleset based on fuzzy concepts, wherein the cerebrovascular accident ruleset represents clinician expertise data associated with cerebrovascular accident procedures for a patient population;
initialize a fuzzy machine learning model based on the cerebrovascular accident ruleset;
train the fuzzy machine learning model on historical electronic health record (EHR) data for the patient population to generate a trained fuzzy ruleset;
generate, via one or more trained autoencoders, at least one embedding vector for medical imaging data of a patient;
generate contextual information for the medical imaging data based on the historical EHR data for the patient population; and
apply the trained fuzzy ruleset to the at least one embedding vector and the contextual information to generate one or more of: (i) an intervention recommendation for the patient, and (ii) a stroke outcome classification for the medical imaging data.

12. The computing system of claim 11, the one or more memories further including computer-executable instructions that, when executed by the one or more processors, train the fuzzy machine learning model on historical EHR data for the patient population to generate a trained fuzzy ruleset by causing the computing system to:
train, using the fuzzy machine learning model, the cerebrovascular accident ruleset based on the historical EHR data.

13. The computing system of claim 12, the one or more memories further including computer-executable instructions that, when executed by the one or more processors, train the cerebrovascular accident ruleset based on the historical EHR data by causing the computing system to:
approximate, using tropical geometry, a continuous representation of a piecewise categorizing function, and
generate, based on at least the continuous representation of the piecewise categorizing function, the trained fuzzy ruleset.

14. The computing system of claim 11, the one or more memories further including computer-executable instructions that, when executed by the one or more processors, cause the computing system to:
encode the clinician expertise data into fuzzy concepts, wherein the fuzzy concepts represent approximate logical relationships between variables.

15. The computing system of claim 11, wherein the historical EHR data includes partially available privileged information for the patient population, and
the one or more memories further including computer-executable instructions that, when executed by the one or more processors, cause the computing system to:
train an artificial neural network model to learn a function that maps a standard feature space of historical medical image embeddings and a privileged feature space of the historical EHR data to an outcome.

16. The computing system of claim 11, the one or more memories further including computer-executable instructions that, when executed by the one or more processors, cause the computing system to:
train at least one autoencoder of the one or more autoencoders on a historical medical imaging dataset to reduce dimensionality of input feature vectors,
wherein imaging data included in the historical medical imaging dataset is mapped to a standardized coordinate system and normalized by applying a brain mask.

17. The computing system of claim 11, wherein the stroke outcome classification is a length of stay (LOS) classification, a modified Rankin scale (mRS) classification, a recovery timeline classification, or an intervention classification.

18. The computing system of claim 11, wherein the medical imaging data is magnetic resonance imaging (MRI) data, computed tomography (CT) imaging data, or computed tomography angiography (CTA) imaging data.

19. The computing system of claim 11, the one or more memories further including computer-executable instructions that, when executed by the one or more processors, cause the computing system to:
present, on a display of a user computing device, the intervention recommendation, or the stroke outcome classification.

20. The computing system of claim 19, the one or more memories further including computer-executable instructions that, when executed by the one or more processors, cause the computing system to:
present, on a display of a user computing device, one or more first indications of corresponding rules included in the trained fuzzy ruleset.
